(19) United States
(12) Patent Application Publication
(10) Pub. No.: US 2010/0064392 A1
(43) Pub. Date: Mar. 11, 2010

(54) NUCLEOTIDE SEQUENCES AND CORRESPONDING POLYPEPTIDES CONFERRING IMPROVED AGRICULTURAL AND/OR ORNAMENTAL CHARACTERISTICS TO PLANTS BY MODULATING ABSCISSION

(75) Inventors: Dennis YANG, Creve Coeur, MO (US); Wuyi Wang, Newbury Park, CA (US)

Correspondence Address: BIRCH STEWART KOLASCH & BIRCH, PO BOX 747, FALLS CHURCH, VA, 22040-0747, US

(73) Assignee: CERES, INC., Thousand Oaks, CA (US)

(21) Appl. No.: 12/481,338

(22) Filed: Jun. 9, 2009

Related U.S. Application Data
(60) Provisional application No. 61/060,194, filed on Jun. 10, 2008.

(51) Int. Cl. A01H 5/00; C12N 15/82; C12N 5/04; C07H 21/00

(52) U.S. Cl. 800/290; 800/298; 800/320; 800/320.1; 800/312; 800/306; 800/320.3; 800/314; 800/320.2; 800/322; 435/419; 536/23.1

(57) ABSTRACT

Methods and materials for modulating abscission and/or inflorescence development time levels in plants are disclosed. For example, nucleic acids encoding abscission and/or inflorescence development time-modulating polypeptides are disclosed as well as methods for using such nucleic acids to transform plant cells. Also disclosed are plants having modulated abscission and/or inflorescence development time levels and plant products produced from plants having such levels.

This Nonprovisional application claims priority under 35 U.S.C. § 119(e) on U.S. Provisional Application No. 61/060,194 filed on Jun. 10, 2008, the entire contents of which are hereby incorporated by reference.

### INCORPORATION-BY-REFERENCE OF SEQUENCE LISTING OR TABLE

The material in the accompanying sequence listing is hereby incorporated by reference into this application. The accompanying file, named ‘2008-04-28 sequence_listingMSDOS.txt’ was created on Apr. 16, 2008 and is 95 KB. The file can be accessed using Microsoft Word on a computer that uses Windows OS.

### TECHNICAL FIELD

This document relates to methods and materials involved in modulating abscission and/or inflorescence development time in plants. The present invention further relates to using nucleic acid molecules and polypeptides to make transgenic plants, plant cells, plant materials or seeds of a plant having improved agricultural and/or ornamental characteristics due to the modulation of abscission and/or inflorescence development time, as compared to wild-type plants.

### BACKGROUND

Plants specifically improved for agriculture, horticulture, biomass conversion, and other industries (e.g. paper industry, plants as production factories for proteins or other compounds) can be obtained using molecular technologies. As an example, great agricultural and ornamental value can result from modulating abscission in plants.

Abscission refers to the process by which a plant intentionally drops one or more of its parts or organs. One model for inducing abscission provides that auxin renders cells in the abscission zone insensitive to ethylene, thereby preventing abscission. Upon auxin concentration dropping below a threshold level (likely set by relative ethylene concentrations), cells in the abscission zone perceive ethylene as an abscission-inducing signal. A person of ordinary skill in the art will recognize many other models of abscission.

Abscission may be induced by environmental and/or developmental events. For instance, upon a plant experiencing an environmental cue, such as shortened daylight, auxin production in a fruit or leaf may drop, causing less auxin to translocate through the abscission zone; thereby allowing ethylene to induce abscission. In addition, a wounded plant may produce high levels of ethylene at the site of the injury; thereby inducing abscission. A person of ordinary skill in the art will recognize many other environmental and developmental cues may induce or inhibit abscission.

Once the abscission pathway is activated, several well documented molecular and physiological events occur prior to the dropping of a plant part or organ. For instance, activation of the abscission pathway results in the expression and/or activation of cell wall degrading enzymes, such as pectinases and cellulases, which structurally weaken the plant part or organ to be dropped. While several nucleic acids and proteins involved in activating and effecting the abscission pathway have been identified, others are either currently under investigation or yet to be discovered.

Abscission is an important process in agriculturally and ornamentally valuable plants. It follows that the modulation of abscission, whether for the purpose of inducing or inhibiting the dropping of a plant part or organ, has important and wide-ranging applications. For instance, the flowering shelf-life of ornamental plants may be extended by inhibiting abscission. In both agricultural and ornamental plants, delayed leaf and/or petal detachment via inhibiting abscission would lead to prolonged photosynthetic activity and/or retention of leaves that will generate cultivars of increased biomass and improved crop and flower yield.

In addition, optimal crop set, maturation, flavoring, coloring, harvesting, sugar development, etc. in a variety of agriculturally important crops may be obtained by modulating abscission. For example, a variety of crops are dependent on bee pollination to set a crop, but flower only briefly due to abscission mediated flower drop soon after blossom. If conditions during blossom are cold, rainy or otherwise unfavorable to bee pollination, a light crop may be set as a result of low pollination rate. In this context, the inhibition of abscission would allow for blossoms to remain on the plant for an extended period of time (i.e. into favorable pollination conditions), and thereby provide for a favorably set crop.

Further agricultural applications for modulating abscission arise in the context of controlling the extent and timing of abscission which occurs naturally over the course of cultivating a set crop to maturity. In particular, many agriculturally important plants go through periods of heightened abscission activity in a relatively short time frame between pollination and maturation of a crop which leads to the dropping of the crop (e.g. fruits, nuts, etc.). In this context, both positive and negative modulation of abscission, at appropriate times, may prove advantageous.

For instance, it is well known that crops set in a given agricultural orchard, grove, vineyard, field, etc. can vary significantly from season to season for reasons including, but not limited to, pollination efficiency, inhospitable whether, alternate bearing effects, etc. In years of light crop set, it may prove advantageous to reduce abscission in order to prevent immature crop-drop; thereby increasing the amount of fruit brought to maturity and market.

In years of excessive crop set, a given agricultural orchard, grove, vineyard, field, etc. will often produce a crop of great quantity, which is undersized and/or has sub-optimally flavor, color, sugar content, etc. In this context, increasing abscission would thin the crop, and lead to the maturation and marketing of a crop lesser in quantity, but more economically valuable due to improved size, flavor, color, sugar content, etc.

According to similar principles, a person of ordinary skill in the art will recognize that, in years of optimal crop set, the extent of naturally occurring immature abscission mediated crop-drop may be either excessive or insufficient. In such circumstances, it is further recognized that appropriately modulating abscission may prove advantageous.

The ability to induce abscission could also prove advantageous in the context cultivating a crop which has matured. For example, inducing abscission may facilitate harvest by weakening the attachment of crop product to crop plant, making it easier to hand pick or mechanically harvest the crop. Moreover, appropriately modulating post-maturation crop abscission would allow for the timing of harvest to coincide with a period of favorable market price.

A person of ordinary skill in the art will recognize the sequence of first inhibiting crop abscission followed by inducing it would provide even tighter control of harvest timing. In addition to market timing, the ability to control abscission in crops capable of harvest would provide the ability to control color development, sugar content, flavor, etc. in order to obtain a favorable market price.

The availability and sustainability of a stream of food and feed for people and domesticated animals has been a high priority throughout the history of human civilization and lies at the origin of agriculture. Specialists and researchers in the fields of agronomy science, agriculture, crop science, horticulture and forest science are even today constantly striving to find and produce plants with an increased growth potential to feed an increasing world population and to guarantee a supply of reproducible raw materials. The robust level of research in these fields of science indicates the level of importance leaders in every geographic environment and climate around the world place on providing sustainable sources of food, feed and energy.

Manipulation of crop performance has been accomplished conventionally for centuries through selection and plant breeding. The breeding process is, however, both time-consuming and labor-intensive. Furthermore, appropriate breeding programs must be specially designed for each relevant plant species.

On the other hand, great progress has been made in using molecular genetic approaches to manipulate plants to provide better crops. Through the introduction and expression of recombinant nucleic acid molecules in plants, researchers are now poised to provide the community with plant species tailored to grow more efficiently and yield more product despite suboptimal geographic and/or climatic environments. These new approaches have the additional advantage of not being limited to one plant species, but instead being applicable to multiple different plant species (Zhang et al. (2004) Plant Physiol. 135:615; Zhang et al. (2001) Proc. Natl. Acad. Sci. USA 98:12832).

Despite this progress, today there continues to be a great need for generally applicable processes that improve forest or agricultural plant growth to suit particular needs depending on specific environmental conditions. To this end, the present invention is directed to advantageously manipulating abscission in plants in order to maximize the benefits of various crops depending on the benefit sought, and is characterized by expression of recombinant DNA molecules in plants. These molecules may be from the plant itself, and simply expressed at a higher or lower level, or the molecules may be from different plant species.

### SUMMARY OF THE INVENTION

This document provides methods and materials related to plants having modulated abscission and/or inflorescence development time (such as, but not limited to, modulated petal abscission, modulated uniformity of flowering time of one or more inflorescences of a plant, and/or modulated duration of flowering time of one or more inflorescences of a plant). For example, this document provides transgenic plants and plant cells having modulated tissue abscission time, nucleic acids used to generate transgenic plants and plant cells having modulated tissue abscission time, and methods for making plants and plant cells having modulated tissue abscission time, modulated uniformity of flowering time of one or more inflorescences of a plant, and/or modulated duration of flowering time of one or more inflorescences of a plant. Such plants and plant cells can be grown to produce, for example, crop plants and ornamental plants having delayed petal abscission. Crop plants having delayed petal abscission levels may be useful to increase pollination rate and yield. Ornamental plants having delayed petal abscission levels may be useful to produce more valuable ornamental plants. Crop and/or ornamental plants having increased uniformity of flowering time of one or more inflorescences of a plant may be useful for more uniform maturation and optimization of yield for a harvest. Crop and/or ornamental plants having increased duration of flowering time may be useful to increase the potential number of flowers per inflorescence and enhance yield. An increase in duration of flowering time may correlate to delayed inflorescence maturity. Plants having delayed inflorescence maturity may have increased biomass levels or increased biomass levels of non-reproductive tissues either of which may be useful in converting such biomass to a liquid fuel or other chemicals, or may be useful as a thermochemical fuel.

Methods of producing a plant are provided herein. In one aspect, a method comprises growing a plant cell comprising an exogenous nucleic acid. The exogenous nucleic acid comprises a regulatory region operably linked to a nucleotide sequence encoding a polypeptide. The Hidden Markov Model (HMM) bit score of the amino acid sequence of the polypeptide is greater than about 115 or 200, using an HMM generated from the amino acid sequences depicted in one of FIG. 1 or 2, respectively. The plant has a difference in the level of tissue abscission and/or inflorescence development time as compared to the corresponding level of a control plant that does not comprise the exogenous nucleic acid.

In another aspect, a method comprises growing a plant cell comprising an exogenous nucleic acid. The exogenous nucleic acid comprises a regulatory region operably linked to a nucleotide sequence encoding a polypeptide having 80 percent or greater sequence identity to an amino acid sequence set forth in SEQ ID NOs: 3, 4, 6, 8, 10, 11, 12, 14, 16, 18, 19, 21, 22, 24, 25, 26, 27, 28, 31, 32, 34, 36, 38, 40, 42, 44, 46, 47, 49, 51, 53, 55, 57, 59, 61, 63, 65, 67, or 69.

A plant produced from the plant cell has a difference in the level of tissue abscission and/or inflorescence development time as compared to the corresponding level of a control plant that does not comprise the exogenous nucleic acid.

In another aspect, a method comprises growing a plant cell comprising an exogenous nucleic acid. The exogenous nucleic acid comprises a regulatory region operably linked to a nucleotide sequence having 80 percent or greater sequence identity to a nucleotide sequence, or a fragment thereof, set forth in SEQ ID NO: 1, 2, 5, 7, 9, 13, 15, 17, 20, 23, 29, 30, 33, 35, 37, 39, 41, 43, 45, 48, 50, 52, 54, 56, 58, 60, 62, 64, 66, or 68. A plant produced from the plant cell has a difference in the level of tissue abscission and/or inflorescence development time as compared to the corresponding level of a control plant that does not comprise the exogenous nucleic acid.

Methods of modulating the tissue abscission, uniformity of flowering time of one or more inflorescences of a plant, and/or duration of flowering time of one or more inflorescences of a plant in a plant are provided herein. In one aspect, a method comprises introducing into a plant cell an exogenous nucleic acid, that comprises a regulatory region operably linked to a nucleotide sequence encoding a polypeptide. The HMM bit score of the amino acid sequence of the polypeptide is greater than about 115, using an HMM generated from the amino acid sequences depicted in one of FIG. 1 or 2. A plant produced from the plant cell has a difference in the level of tissue abscission and/or inflorescence development time as compared to the corresponding level of a control plant that does not comprise the exogenous nucleic acid.

In certain embodiments, the amino acid sequence of the polypeptide is greater than about 40, using an HMM generated from the amino acid sequences depicted in FIG. 2, wherein the polypeptide comprises an Harpin-induced protein 1 (Hin1) domain having 85 percent or greater sequence identity to residues 74 to 213 OF SEQ ID NO: 3, residues 65 to 208 OF SEQ ID NO: 4, residues 58 to 196 OF SEQ ID NO: 6, residues 34 to 174 OF SEQ ID NO: 8, residues 31 to 170 OF SEQ ID NO: 10, residues 31 to 170 OF SEQ ID NO: 11, residues 61 to 204 OF SEQ ID NO: 12, residues 62 to 204 OF SEQ ID

NO: 14, residues 89 to 229 OF SEQ ID NO: 16, residues 35 to 175 OF SEQ ID NO: 18, residues 59 to 198 OF SEQ ID NO: 19, residues 65 to 207 OF SEQ ID NO: 21, residues 71 to 213 OF SEQ ID NO: 22, residues 58 to 178 OF SEQ ID NO: 24, residues 63 to 208 OF SEQ ID NO: 25, residues 74 to 213 OF SEQ ID NO: 26, residues 71 to 213 OF SEQ ID NO: 27, or residues 69 to 211 OF SEQ ID NO: 28.

In another aspect, a method comprises introducing into a plant cell an exogenous nucleic acid that comprises a regulatory region operably linked to a nucleotide sequence encoding a polypeptide having 80 percent or greater sequence identity to an amino acid sequence set forth in SEQ ID NO: 3, 4, 6, 8, 10, 11, 12, 14, 16, 18, 19, 21, 22, 24, 25, 26, 27, 28, 31, 32, 34, 36, 38, 40, 42, 44, 46, 47, 49, 51, 53, 55, 57, 59, 61, 63, 65, 67, or 69. A plant produced from the plant cell has a difference in the level of tissue abscission and/or inflorescence development time as compared to the corresponding level of a control plant that does not comprise the exogenous nucleic acid.

In another aspect, a method comprises introducing into a plant cell an exogenous nucleic acid, that comprises a regulatory region operably linked to a nucleotide sequence having 80 percent or greater sequence identity to a nucleotide sequence set forth in SEQ ID NO: 1, 2, 5, 7, 9, 13, 15, 17, 20, 23, 29, 30, 33, 35, 37, 39, 41, 43, 45, 48, 50, 52, 54, 56, 58, 60, 62, 64, 66, 68, or a fragment thereof. A plant produced from the plant cell has a difference in the level of tissue abscission and/or inflorescence development time as compared to the corresponding level of a control plant that does not comprise the exogenous nucleic acid.

Plant cells comprising an exogenous nucleic acid are provided herein. In one aspect, the exogenous nucleic acid comprises a regulatory region operably linked to a nucleotide sequence encoding a polypeptide. The HMM bit score of the amino acid sequence of the polypeptide is greater than about 115, using an HMM based on the amino acid sequences depicted in one of FIG. 1 or 2. The plant has a difference in the level of tissue abscission and/or inflorescence development time as compared to the corresponding level of a control plant that does not comprise the exogenous nucleic acid. In another aspect, the exogenous nucleic acid comprises a regulatory region operably linked to a nucleotide sequence encoding a polypeptide having 80 percent or greater sequence identity to an amino acid sequence selected from the group consisting of SEQ ID NO: 3, 4, 6, 8, 10, 11, 12, 14, 16, 18, 19, 21, 22, 24, 25, 26, 27, 28, 31, 32, 34, 36, 38, 40, 42, 44, 46, 47, 49, 51, 53, 55, 57, 59, 61, 63, 65, 67, or 69. A plant produced from the plant cell has a difference in the level of tissue abscission and/or inflorescence development time as compared to the corresponding level of a control plant that does not comprise the exogenous nucleic acid. In another aspect, the exogenous nucleic acid comprises a regulatory region operably linked to a nucleotide sequence having 80 percent or greater sequence identity to a nucleotide sequence selected from the group consisting of SEQ ID NO: 1, 2, 5, 7, 9, 13, 15, 17, 20, 23, 29, 30, 33, 35, 37, 39, 41, 43, 45, 48, 50, 52, 54, 56, 58, 60, 62, 64, 66, 68, or a fragment thereof. A plant produced from the plant cell has a difference in the level of tissue abscission and/or inflorescence development time as compared to the corresponding level of a control plant that does not comprise the exogenous nucleic acid. A transgenic plant comprising such a plant cell is also provided. Also provided is a seed and/or fruit product. The product comprises embryonic tissue from a transgenic plant. Also provided is a flower product. The product comprises reproductive tissue from a transgenic plant. Also provided is a biomass product. The product comprises tissue from a transgenic plant.

Isolated nucleic acids are also provided. In one aspect, an isolated nucleic acid comprises a nucleotide sequence having 85% or greater sequence identity to the nucleotide sequence set forth in SEQ ID NO: 1, 2, 5, 7, 9, 13, 15, 17, 20, 23, 29, 30, 33, 35, 37, 39, 41, 43, 45, 48, 50, 52, 54, 56, 58, 60, 62, 64, 66, or 68. In another aspect, an isolated nucleic acid comprises a nucleotide sequence encoding a polypeptide having 80% or greater sequence identity to the amino acid sequence set forth in SEQ ID NO: 3, 4, 6, 8, 10, 11, 12, 14, 16, 18, 19, 21, 22, 24, 25, 26, 27, 28, 31, 32, 34, 36, 38, 40, 42, 44, 46, 47, 49, 51, 53, 55, 57, 59, 61, 63, 65, 67, or 69.

In another aspect, methods of identifying a genetic polymorphism associated with variation in the level of tissue abscission, uniformity of flowering time of one or more inflorescences of a plant, and/or duration of flowering time of one or more inflorescences of a plant are provided. The methods include providing a population of plants, and determining whether one or more genetic polymorphisms in the population are genetically linked to the locus for a polypeptide selected from the group consisting of the polypeptides depicted in FIG. 1 or 2 and functional homologs thereof. The correlation between variation in the level of tissue abscission and/or inflorescence development time in a tissue in plants of the population and the presence of the one or more genetic polymorphisms in plants of the population is measured, thereby permitting identification of whether or not the one or more genetic polymorphisms are associated with such variation.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims. The word “comprising” in the claims may be replaced by “consisting essentially of” or by “consisting of,” according to standard practice in patent law.

### BRIEF DESCRIPTION OF THE DRAWINGS

FIG. 1 is an alignment of the amino acid sequence of ME03564 (SEQ ID NO: 31 which corresponds to Ceres Clone 1678; Ceres cDNA ID 23364915; and locus identifier At3g54200) with homologous and/or orthologous amino acid sequences. In all the alignment figures shown herein, a dash in an aligned sequence represents a gap, i.e., a lack of an amino acid at that position. Identical amino acids or conserved amino acid substitutions among aligned sequences are identified by boxes. FIG. 1 and the other alignment figures provided herein were generated using the program MUSCLE version 3.52.

FIG. 2 is an alignment of the amino acid sequence of ME05885 (SEQ ID NO: 3 which corresponds to Ceres Clone 32430) with homologous and/or orthologous amino acid sequences.

### DETAILED DESCRIPTION OF THE INVENTION

The invention features methods and materials related to modulating levels of tissue abscission in plants, uniformity of flowering time of one or more inflorescences of a plant, and/or duration of flowering time of one or more inflorescences of a plant. In some embodiments, the plants may also have modulated levels of biomass. The methods can include transforming a plant cell with a nucleic acid encoding a tissue abscission and/or inflorescence development time-modulating polypeptide, wherein expression of the polypeptide results in a modulated level of tissue abscission. Plant cells produced using such methods can be grown to produce plants having an increased or decreased tissue abscission. Such plants, and the seeds of such plants, may be used to produce, for example, plants having a delayed floral abscission, increased uniformity of flowering time of one or more inflorescences of a plant, and/or increased duration of flowering time of one or more inflorescences of a plant. Such plants and parts thereof are useful as ornamental products that have altered flower development timing, agricultural products having increased yields, or as biomass useful for conversion to fuel.

### I. Definitions

“Amino acid” refers to one of the twenty biologically occurring amino acids and to synthetic amino acids, including D/L optical isomers.

“Cell type-preferential promoter” or “tissue-preferential promoter” refers to a promoter that drives expression preferentially in a target cell type or tissue, respectively, but may also lead to some transcription in other cell types or tissues as well.

“Control plant” refers to a plant that does not contain the exogenous nucleic acid present in a transgenic plant of interest, but otherwise has the same or similar genetic background as such a transgenic plant. A suitable control plant can be a non-transgenic wild type plant, a non-transgenic segregant from a transformation experiment, or a transgenic plant that contains an exogenous nucleic acid other than the exogenous nucleic acid of interest.

“Domains” are groups of substantially contiguous amino acids in a polypeptide that can be used to characterize protein families and/or parts of proteins. Such domains have a “fingerprint” or “signature” that can comprise conserved primary sequence, secondary structure, and/or three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

“Down-regulation” refers to regulation that decreases production of expression products (mRNA, polypeptide, or both) relative to basal or native states.

“Exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

“Expression” refers to the process of converting genetic information of a polynucleotide into RNA through transcription, which is catalyzed by an enzyme, RNA polymerase, and into protein, through translation of mRNA on ribosomes.

“Heterologous polypeptide” as used herein refers to a polypeptide that is not a naturally occurring polypeptide in a plant cell, e.g., a transgenic Panicum virgatum plant transformed with and expressing the coding sequence for a nitrogen transporter polypeptide from a Zea mays plant.

“Isolated nucleic acid” as used herein includes a naturally-occurring nucleic acid, provided one or both of the sequences immediately flanking that nucleic acid in its naturally-occurring genome is removed or absent. Thus, an isolated nucleic acid includes, without limitation, a nucleic acid that exists as a purified molecule or a nucleic acid molecule that is incorporated into a vector or a virus. A nucleic acid existing among hundreds to millions of other nucleic acids within, for example, cDNA libraries, genomic libraries, or gel slices containing a genomic DNA restriction digest, is not to be considered an isolated nucleic acid.

“Modulation” of the level of tissue abscission, uniformity of flowering time of one or more inflorescences of a plant, and/or duration of flowering time of one or more inflorescences of a plant refers to the change in the level of the indicated trait that is observed as a result of expression of, or transcription from, an exogenous nucleic acid in a plant cell. The change in level is measured relative to the corresponding level in control plants.

“Nucleic acid” and “polynucleotide” are used interchangeably herein, and refer to both RNA and DNA, including cDNA, genomic DNA, synthetic DNA, and DNA or RNA containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, nucleic acid probes and nucleic acid primers. A polynucleotide may contain unconventional or modified nucleotides.

“Operably linked” refers to the positioning of a regulatory region and a sequence to be transcribed in a nucleic acid so that the regulatory region is effective for regulating transcription or translation of the sequence. For example, to operably link a coding sequence and a regulatory region, the translation initiation site of the translational reading frame of the coding sequence is typically positioned between one and about fifty nucleotides downstream of the regulatory region. A regulatory region can, however, be positioned as much as about 5,000 nucleotides upstream of the translation initiation site, or about 2,000 nucleotides upstream of the transcription start site.

“Polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition.

“Progeny” includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC1, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989).

“Up-regulation” refers to regulation that increases the level of an expression product (mRNA, polypeptide, or both) relative to basal or native states.

“Vector” refers to a replicon, such as a plasmid, phage, or cosmid, into which another DNA segment may be inserted so as to bring about the replication of the inserted segment. Generally, a vector is capable of replication when associated with the proper control elements. The term “vector” includes cloning and expression vectors, as well as viral vectors and integrating vectors. An “expression vector” is a vector that includes a regulatory region.

### II. Polypeptides

Polypeptides described herein include tissue abscission and/or inflorescence development time-modulating polypeptides. Tissue abscission and/or inflorescence development time-modulating polypeptides can be effective to modulate tissue abscission levels and/or inflorescence development time when expressed in a plant or plant cell. Such polypeptides typically contain at least one domain indicative of tissue abscission and/or inflorescence development time-modulating polypeptides, as described in more detail herein. Tissue abscission and/or inflorescence development time-modulating polypeptides typically have an HMM bit score that is greater than 115, as described in more detail herein. In some embodiments, tissue abscission and/or inflorescence development time-modulating polypeptides have greater than 20% identity to SEQ ID NOs: 3, 4, 6, 8, 10, 11, 12, 14, 16, 18, 19, 21, 22, 24, 25, 26, 27, 28, 31, 32, 34, 36, 38, 40, 42, 44, 46, 47, 49, 51, 53, 55, 57, 59, 61, 63, 65, 67, or 69, as described in more detail herein.

A. Domains Indicative of Tissue Abscission and/or Inflorescence Development Time-Modulating Polypeptides

A tissue abscission and/or inflorescence development time-modulating polypeptide can contain a Hin1 domain, which is predicted to be characteristic of an tissue abscission and/or inflorescence development time-modulating polypeptide polypeptide. SEQ ID NO: 3 sets forth the amino acid sequence of an Arabidopsis clone, identified herein as CeresClone:1678 (SEQ ID NO: 3), that is predicted to encode a polypeptide containing a Hin1 domain

In certain embodiments, the amino acid sequence of the polypeptide comprises an Hin1 domain having 50, 60, 70, 80, 85, 90, 95, 96, 97, 98 or 99 percent or greater sequence identity to residues 74 to 213 OF SEQ ID NO: 3, residues 65 to 208 OF SEQ ID NO: 4, residues 58 to 196 OF SEQ ID NO: 6, residues 34 to 174 OF SEQ ID NO: 8, residues 31 to 170 OF SEQ ID NO: 10, residues 31 to 170 OF SEQ ID NO: 11, residues 61 to 204 OF SEQ ID NO: 12, residues 62 to 204 OF SEQ ID NO: 14, residues 89 to 229 OF SEQ ID NO: 16, residues 35 to 175 OF SEQ ID NO: 18, residues 59 to 198 OF SEQ ID NO: 19, residues 65 to 207 OF SEQ ID NO: 21, residues 71 to 213 OF SEQ ID NO: 22, residues 58 to 178 OF SEQ ID NO: 24, residues 63 to 208 OF SEQ ID NO: 25, residues 74 to 213 OF SEQ ID NO: 26, residues 71 to 213 OF SEQ ID NO: 27, or residues 69 to 211 OF SEQ ID NO: 28.

### B. Functional Homologs Identified by Reciprocal BLAST

In some embodiments, one or more functional homologs of a reference tissue abscission and/or inflorescence development time-modulating polypeptide defined by one or more of the Pfam descriptions indicated above are suitable for use as tissue abscission and/or inflorescence development time-modulating polypeptides. A functional homolog is a polypeptide that has sequence similarity to a reference polypeptide, and that carries out one or more of the biochemical or physiological function(s) of the reference polypeptide. A functional homolog and the reference polypeptide may be natural occurring polypeptides, and the sequence similarity may be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, may themselves be functional homologs. Functional homologs can also be created via site-directed mutagenesis of the coding sequence for a tissue abscission and/or inflorescence development time-modulating polypeptide, or by combining domains from the coding sequences for different naturally-occurring tissue abscission and/or inflorescence development time-modulating polypeptides (“domain swapping”). The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of tissue abscission and/or inflorescence development time-modulating polypeptides. Sequence analysis can involve BLAST, Reciprocal BLAST, or PSI-BLAST analysis of nonredundant databases using a tissue abscission and/or inflorescence development time-modulating polypeptide amino acid sequence as the reference sequence. Amino acid sequence is, in some instances, deduced from the nucleotide sequence. Those polypeptides in the database that have greater than 40% sequence identity are candidates for further evaluation for suitability as a tissue abscission and/or inflorescence development time-modulating polypeptide. Amino acid sequence similarity allows for conservative amino acid substitutions, such as substitution of one hydrophobic residue for another or substitution of one polar residue for another. Conservative amino acid substitutions generally refer to a substitution of an amino acid with another amino acid having similar chemical properties. The best operational definition for a conservative amino acid substitution is replacement of one amino acid with another amino acid with a low substitution penalty in the scoring matrix used and these are well known and described in the art, for example in Henikoff, S. and Henikoff, J. Proc. Natl. Acad. Sci. USA. 89: 10915-10919 (1992), which is herein incorporated by reference in its entirety.

If desired, manual inspection of such candidates can be carried out in order to narrow the number of candidates to be further evaluated. Manual inspection can be performed by selecting those candidates that appear to have domains present in tissue abscission and/or inflorescence development time-modulating polypeptides, e.g., conserved functional domains.

Conserved regions can be identified by locating a region within the primary amino acid sequence of a tissue abscission and/or inflorescence development time-modulating polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, at least 85% or at least 90% amino acid sequence identity). In some embodiments, a conserved region exhibits at least 92%, 94%, 95%, 96%, 98%, or 99% amino acid sequence identity.

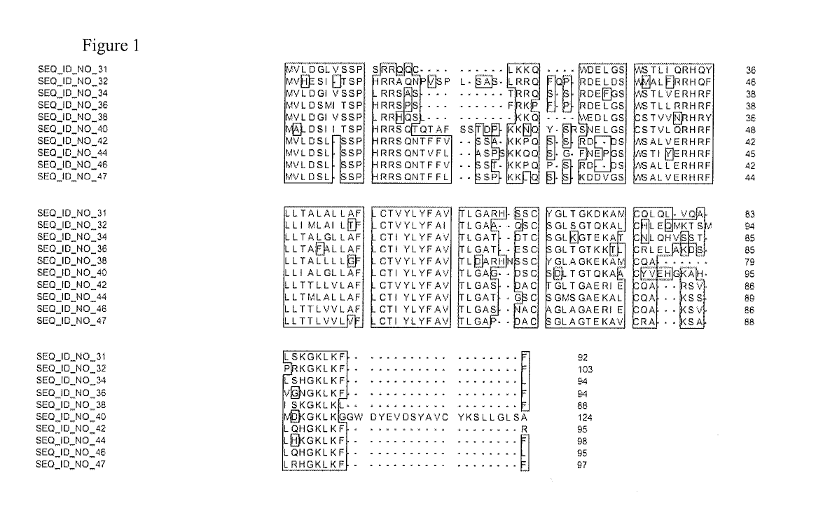

Examples of amino acid sequences of functional homologs of the polypeptide set forth in SEQ ID NO: 31 are provided in FIG. 1 and in the Sequence Listing. Such functional homologs include CeresClone:32430 (SEQ ID NO: 31). In some cases, a functional homolog of SEQ ID NO: 31 has an amino acid sequence with at least 20% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 31.

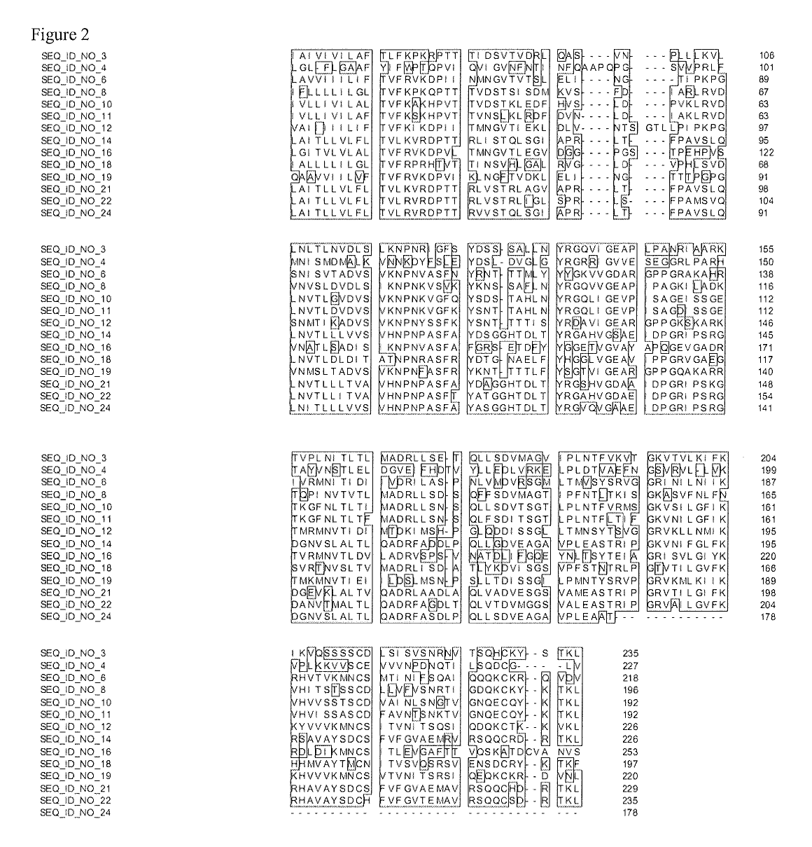

Examples of amino acid sequences of functional homologs of the polypeptide set forth in SEQ ID NO: 3 are provided in FIG. 2 and in the Sequence Listing. Such functional homologs include CeresClone:1678 (SEQ ID NO: 3). In some cases, a functional homolog of SEQ ID NO: 3 has an amino acid sequence with at least 20% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 3.

The identification of conserved regions in a tissue abscission and/or inflorescence development time-modulating polypeptide facilitates production of variants of tissue abscission and/or inflorescence development time-modulating polypeptides. Variants of tissue abscission and/or inflorescence development time-modulating polypeptides typically have 10 or fewer conservative amino acid substitutions within the primary amino acid sequence, e.g., 7 or fewer conservative amino acid substitutions, 5 or fewer conservative amino acid substitutions, or between 1 and 5 conservative substitutions. A useful variant polypeptide can be constructed based on one of the alignments set forth in FIG. 1 or FIG. 2 and/or homologs identified in the Sequence Listing. Such a polypeptide includes the conserved regions, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

### C. Functional Homologs Identified by HMMER

In some embodiments, useful tissue abscission and/or inflorescence development time-modulating polypeptides include those that fit a Hidden Markov Model based on the polypeptides set forth in any one of FIGS. 1-2. A Hidden Markov Model (HMM) is a statistical model of a consensus sequence for a group of functional homologs. See, Durbin et al., Biological Sequence Analysis: Probabilistic Models of Proteins and Nucleic Acids, Cambridge University Press, Cambridge, UK (1998). An HMM is generated by the program HMMER 2.3.2 with default program parameters, using the sequences of the group of functional homologs as input. The multiple sequence alignment is generated by ProbCons (Do et al., Genome Res., 15(2):330-40 (2005)) version 1.11 using a set of default parameters: -c, --consistency REPS of 2; -ir, --iterative-refinement REPS of 100; -pre, --pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. HMMER 2.3.2 was released Oct. 3, 2003 under a GNU general public license, and is available from various sources on the World Wide Web such as hmmer.janelia.org; hmmer.wustl.edu; and fr.com/hmmer232/. Hmmbuild outputs the model as a text file.

The HMM for a group of functional homologs can be used to determine the likelihood that a candidate tissue abscission and/or inflorescence development time-modulating polypeptide sequence is a better fit to that particular HMM than to a null HMM generated using a group of sequences that are not structurally or functionally related. The likelihood that a candidate polypeptide sequence is a better fit to an HMM than to a null HMM is indicated by the HMM bit score, a number generated when the candidate sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the candidate sequence carries out one or more of the biochemical or physiological function(s) of the polypeptides used to generate the HMM. A high HMM bit score is at least 20, and often is higher. Slight variations in the HMM bit score of a particular sequence can occur due to factors such as the order in which sequences are processed for alignment by multiple sequence alignment algorithms such as the ProbCons program. Nevertheless, such HMM bit score variation is minor.

The tissue abscission and/or inflorescence development time-modulating polypeptides discussed below fit the indicated HMM with an HMM bit score greater than 20 (e.g., greater than 30, 40, 50, 60, 70, 80, 90, 100, 200, 300, 400, or 500). In some embodiments, the HMM bit score of a tissue abscission and/or inflorescence development time-modulating polypeptide discussed below is about 50%, 60%, 70%, 80%, 90%, or 95% of the HMM bit score of a functional homolog provided the Sequence Listing of this application. In some embodiments, a tissue abscission and/or inflorescence development time-modulating polypeptide discussed below fits the indicated HMM with an HMM bit score greater than 20, and has a domain indicative of a tissue abscission and/or inflorescence development time-modulating polypeptide. In some embodiments, a tissue abscission and/or inflorescence development time-modulating polypeptide discussed below fits the indicated HMM with an HMM bit score greater than 20, and has 20% or greater sequence identity (e.g., 75%, 80%, 85%, 90%, 95%, or 100% sequence identity) to an amino acid sequence shown in any one of FIGS. 1-2.

Examples of polypeptides are shown in the sequence listing that have HMM bit scores greater than 115 when fitted to an HMM generated from the amino acid sequences set forth in FIG. 1 are identified in the Sequence Listing of this application. Such polypeptides include CeresClone:6932 (SEQ ID NO: 69), CeresClone:651757 (SEQ ID NO: 67), CeresClone:724313 (SEQ ID NO: 65), CeresClone:1246352 (SEQ ID NO: 63), CeresClone:1116694 (SEQ ID NO: 61), CeresClone:951894 (SEQ ID NO: 59), CeresClone:1246448 (SEQ ID NO: 38), CeresClone:1661096 (SEQ ID NO: 57), CeresAnnot:870132 (SEQ ID NO: 55), CeresAnnot:8706658 (SEQ ID NO: 53), CeresClone:32430 (SEQ ID NO: 31), CeresClone:392748 (SEQ ID NO: 51), CeresAnnot:1501366 (SEQ ID NO: 49), CeresClone:1678697 (SEQ ID NO: 42), CeresClone:1871675 (SEQ ID NO: 46), GI:116788849 (SEQ ID NO: 32), CeresAnnot:1437729 (SEQ ID NO: 36), CeresClone:1808741 (SEQ ID NO: 34), CeresAnnot:8459763 (SEQ ID NO: 40), CeresClone:1727075 (SEQ ID NO: 44), or GI:115475611 (SEQ ID NO: 47).

Examples of polypeptides are shown in the sequence listing that have HMM bit scores greater than 210 when fitted to an HMM generated from the amino acid sequences set forth in FIG. 2 are identified in the Sequence Listing of this application. Such polypeptides include GI:18379145 (SEQ ID NO: 25), CeresClone:2056651 (SEQ ID NO: 24), GI:92896037 (SEQ ID NO: 11), CeresClone:472323 (SEQ ID NO: 10), GI:116782452 (SEQ ID NO: 4), CeresAnnot:1529688 (SEQ ID NO: 8), CeresClone:1731181 (SEQ ID NO: 18), GI:115487056 (SEQ ID NO: 28), GI:115483889 (SEQ ID NO: 22), GI:125533193 (SEQ ID NO: 27), GI:15232445 (SEQ ID NO: 26), CeresClone:1866925 (SEQ ID NO: 6), CeresClone:1469145 (SEQ ID NO: 16), CeresClone:1678 (SEQ ID NO: 3), CeresAnnot:8682708 (SEQ ID NO: 14), CeresClone:1874239 (SEQ ID NO: 21), GI:94549041 (SEQ ID NO: 12), or GI:147832282 (SEQ ID NO: 19).

### D. Percent Identity

In some embodiments, a tissue abscission and/or inflorescence development time-modulating polypeptide has an amino acid sequence with at least 20 percent sequence identity, e.g., 50%, 52%, 56%, 59%, 61%, 65%, 70%, 75%, 80%, 85%, 90%, 95%, 97%, 98%, or 99% sequence identity, to one of the amino acid sequences set forth in SEQ ID NOs: 3, 4, 6, 8, 10, 11, 12, 14, 16, 18, 19, 21, 22, 24, 25, 26, 27, 28, 31, 32, 34, 36, 38, 40, 42, 44, 46, 47, 49, 51, 53, 55, 57, 59, 61, 63, 65, 67, or 69. Polypeptides having such a percent sequence identity often have a domain indicative of a tissue abscission and/or inflorescence development time-modulating polypeptide and/or have an HMM bit score that is greater than 115, as discussed above. Amino acid sequences of tissue abscission and/or inflorescence development time-modulating polypeptides having at least 20% sequence identity to one of the amino acid sequences set forth in SEQ ID NOs: 3 and 31 are provided in FIGS. 1-2 and in the Sequence Listing.

“Percent sequence identity” refers to the degree of sequence identity between any given reference sequence, e.g., SEQ ID NO: 3 or 31, and a candidate tissue abscission and/or inflorescence development time-modulating sequence. A candidate sequence typically has a length that is from 80 percent to 200 percent of the length of the reference sequence, e.g., 82, 85, 87, 89, 90, 93, 95, 97, 99, 100, 105, 110, 115, 120, 130, 140, 150, 160, 170, 180, 190, or 200 percent of the length of the reference sequence. A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chenna et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

In some cases, a tissue abscission and/or inflorescence development time-modulating polypeptide has an amino acid sequence with at least 18% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 31. Amino acid sequences of polypeptides having greater than 18% sequence identity to the polypeptide set forth in SEQ ID NO: 31 are provided in FIG. 1 and in the Sequence Listing. Examples of such poypeptides include CeresClone:6932 (SEQ ID NO: 69), CeresClone:651757 (SEQ ID NO: 67), CeresClone:724313 (SEQ ID NO: 65), CeresClone:1246352 (SEQ ID NO: 63), CeresClone:1116694 (SEQ ID NO: 61), CeresClone:951894 (SEQ ID NO: 59), CeresClone:1246448 (SEQ ID NO: 38), CeresClone:1661096 (SEQ ID NO: 57), CeresAnnot:870132 (SEQ ID NO: 55), CeresAnnot:8706658 (SEQ ID NO: 53), CeresClone:32430 (SEQ ID NO: 31), CeresClone:392748 (SEQ ID NO: 51), CeresAnnot:1501366 (SEQ ID NO: 49), CeresClone:1678697 (SEQ ID NO: 42), CeresClone:1871675 (SEQ ID NO: 46), GI:116788849 (SEQ ID NO: 32), CeresAnnot:1437729 (SEQ ID NO: 36), CeresClone:1808741 (SEQ ID NO: 34), CeresAnnot:8459763 (SEQ ID NO: 40), CeresClone:1727075 (SEQ ID NO: 44), or GI:115475611 (SEQ ID NO: 47).

In some cases, a tissue abscission and/or inflorescence development time-modulating polypeptide has an amino acid sequence with at least 19% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 3. Amino acid sequences of polypeptides having greater than 19% sequence identity to the polypeptide set forth in SEQ ID NO: 3 are provided in FIG. 2 and in the Sequence Listing. Examples of such polypeptides include GI:118379145 (SEQ ID NO: 25), CeresClone:2056651 (SEQ ID NO: 24), GI:92896037 (SEQ ID NO: 11), CeresClone:472323 (SEQ ID NO: 10), GI:116782452 (SEQ ID NO: 4), CeresAnnot:1529688 (SEQ ID NO: 8), CeresClone:1731181 (SEQ ID NO: 18), GI:115487056 (SEQ ID NO: 28), GI:115483889 (SEQ ID NO: 22), GI:125533193 (SEQ ID NO: 27), GI:15232445 (SEQ ID NO: 26), CeresClone:1866925 (SEQ ID NO: 6), CeresClone:1469145 (SEQ ID NO: 16), CeresClone:1678 (SEQ ID NO: 3), CeresAnnot:8682708 (SEQ ID NO: 14), CeresClone:1874239 (SEQ ID NO: 21), GI:94549041 (SEQ ID NO: 12), or GI:147832282 (SEQ ID NO: 19).

### E. Other Sequences

It should be appreciated that a tissue abscission and/or inflorescence development time-modulating polypeptide can include additional amino acids that are not involved in tissue abscission and/or inflorescence development time modulation, and thus such a polypeptide can be longer than would otherwise be the case. For example, a tissue abscission and/or inflorescence development time-modulating polypeptide can include a purification tag, a chloroplast transit peptide, a mitochondrial transit peptide, an amyloplast peptide, or a leader sequence added to the amino or carboxy terminus. In some embodiments, a tissue abscission and/or inflorescence development time-modulating polypeptide includes an amino acid sequence that functions as a reporter, e.g., a green fluorescent protein or yellow fluorescent protein.

### III. Nucleic Acids

Nucleic acids described herein include nucleic acids that are effective to modulate levels of tissue abscission and/or inflorescence development time (such as, but not limited to, uniformity of flowering time of one or more inflorescences of a plant, and/or duration of flowering time of one or more inflorescences of a plant) when transcribed in a plant or plant cell. Such nucleic acids include, without limitation, those that encode a tissue abscission and/or inflorescence development time-modulating polypeptide and those that can be used to inhibit expression of a tissue abscission and/or inflorescence development time-modulating polypeptide via a nucleic acid based method.

A. Nucleic Acids Encoding Tissue Abscission and/or Inflorescence Development Time-Modulating Polypeptides

Nucleic acids encoding tissue abscission and/or inflorescence development time-modulating polypeptides are described herein. Examples of such nucleic acids include SEQ ID NOs: 1, 2, 5, 7, 9, 13, 15, 17, 20, 23, 29, 30, 33, 35, 37, 39, 41, 43, 45, 48, 50, 52, 54, 56, 58, 60, 62, 64, 66, or 68, as described in more detail below. A nucleic acid also can be a fragment that is at least 40% (e.g., at least 45, 50, 55, 60, 65, 70, 75, 80, 85, 90, 95, or 99%) of the length of the full-length nucleic acid set forth in SEQ ID NOs: 1, 2, 5, 7, 9, 13, 15, 17, 20, 23, 29, 30, 33, 35, 37, 39, 41, 43, 45, 48, 50, 52, 54, 56, 58, 60, 62, 64, 66, or 68.

A tissue abscission and/or inflorescence development time-modulating nucleic acid can comprise the nucleotide sequence set forth in SEQ ID NO: 30. Alternatively, a tissue abscission and/or inflorescence development time-modulating nucleic acid can be a variant of the nucleic acid having the nucleotide sequence set forth in SEQ ID NO: 30. For example, a tissue abscission and/or inflorescence development time-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 30.

A tissue abscission and/or inflorescence development time-modulating nucleic acid can comprise the nucleotide sequence set forth in SEQ ID NO: 2. Alternatively, a tissue abscission and/or inflorescence development time-modulating nucleic acid can be a variant of the nucleic acid having the nucleotide sequence set forth in SEQ ID NO: 2. For example, a tissue abscission and/or inflorescence development time-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 2.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

### B. Use of Nucleic Acids to Modulate Expression of Polypeptides

### i. Expression of a Tissue Abscission and/or Inflorescence Development Time-Modulating Polypeptide

A nucleic acid encoding one of the tissue abscission and/or inflorescence development time-modulating polypeptides described herein can be used to express the polypeptide in a plant species of interest, typically by transforming a plant cell with a nucleic acid having the coding sequence for the polypeptide operably linked in sense orientation to one or more regulatory regions. It will be appreciated that because of the degeneracy of the genetic code, a number of nucleic acids can encode a particular tissue abscission and/or inflorescence development time-modulating polypeptide; i.e., for many amino acids, there is more than one nucleotide triplet that serves as the codon for the amino acid. Thus, codons in the coding sequence for a given tissue abscission and/or inflorescence development time-modulating polypeptide can be modified such that optimal expression in a particular plant species is obtained, using appropriate codon bias tables for that species.

In some cases, expression of a tissue abscission and/or inflorescence development time-modulating polypeptide inhibits one or more functions of an endogenous polypeptide. For example, a nucleic acid that encodes a dominant negative polypeptide can be used to inhibit protein function. A dominant negative polypeptide typically is mutated or truncated relative to an endogenous wild type polypeptide, and its presence in a cell inhibits one or more functions of the wild type polypeptide in that cell, i.e., the dominant negative polypeptide is genetically dominant and confers a loss of function. The mechanism by which a dominant negative polypeptide confers such a phenotype can vary but often involves a protein-protein interaction or a protein-DNA interaction. For example, a dominant negative polypeptide can be an enzyme that is truncated relative to a native wild type enzyme, such that the truncated polypeptide retains domains involved in binding a first protein but lacks domains involved in binding a second protein. The truncated polypeptide is thus unable to properly modulate the activity of the second protein. See, e.g., US 2007/0056058. As another example, a point mutation that results in a non-conservative amino acid substitution in a catalytic domain can result in a dominant negative polypeptide. See, e.g., US 2005/032221. As another example, a dominant negative polypeptide can be a transcription factor that is truncated relative to a native wild type transcription factor, such that the truncated polypeptide retains the DNA binding domain(s) but lacks the activation domain(s). Such a truncated polypeptide can inhibit the wild type transcription factor from binding DNA, thereby inhibiting transcription activation.

### ii. Inhibition of Expression of a Tissue Abscission and/or Inflorescence Development Time-Modulating Polypeptide

Polynucleotides and recombinant constructs described herein can be used to inhibit expression of a tissue abscission and/or inflorescence development time-modulating polypeptide in a plant species of interest. See, e.g., Matzke and Birchler, Nature Reviews Genetics 6:24-35 (2005); Akashi et al., Nature Reviews Mol. Cell Biology 6:413-422 (2005); Mittal, Nature Reviews Genetics 5:355-365 (2004); Dorsett and Tuschl, Nature Reviews Drug Discovery 3: 318-329 (2004); and Nature Reviews RNA interference collection, October 2005 at nature.com/reviews/focus/mai. A number of nucleic acid based methods, including antisense RNA, ribozyme directed RNA cleavage, post-transcriptional gene silencing (PTGS), e.g., RNA interference (RNAi), and transcriptional gene silencing (TGS) are known to inhibit gene expression in plants. Suitable polynucleotides include full-length nucleic acids encoding tissue abscission and/or inflorescence development time-modulating polypeptides or fragments of such full-length nucleic acids. In some embodiments, a complement of the full-length nucleic acid or a fragment thereof can be used. Typically, a fragment is at least 10 nucleotides, e.g., at least 12, 13, 14, 15, 16, 17, 18, 19, 20, 21, 22, 23, 24, 25, 26, 27, 30, 35, 40, 50, 80, 100, 200, 500 nucleotides or more. Generally, higher homology can be used to compensate for the use of a shorter sequence.

Antisense technology is one well-known method. In this method, a nucleic acid of a gene to be repressed is cloned and operably linked to a regulatory region and a transcription termination sequence so that the antisense strand of RNA is transcribed. The recombinant construct is then transformed into plants, as described herein, and the antisense strand of RNA is produced. The nucleic acid need not be the entire sequence of the gene to be repressed, but typically will be substantially complementary to at least a portion of the sense strand of the gene to be repressed.

In another method, a nucleic acid can be transcribed into a ribozyme, or catalytic RNA, that affects expression of an mRNA. See, U.S. Pat. No. 6,423,885. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

PTGS, e.g., RNAi, can also be used to inhibit the expression of a gene. For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence or a fragment thereof of a tissue abscission and/or inflorescence development time-modulating polypeptide, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand or a fragment thereof of the coding sequence of the tissue abscission and/or inflorescence development time-modulating polypeptide, and can have a length that is shorter, the same as, or longer than the corresponding length of the sense sequence. In some cases, one strand of the stem portion of a double stranded RNA comprises a sequence that is similar or identical to the 3′ or 5′ untranslated region, or a fragment thereof, of an mRNA encoding a tissue abscission and/or inflorescence development time-modulating polypeptide, and the other strand of the stem portion of the double stranded RNA comprises a sequence that is similar or identical to the sequence that is complementary to the 3′ or 5′ untranslated region, respectively, or a fragment thereof, of the mRNA encoding the tissue abscission and/or inflorescence development time-modulating polypeptide. In other embodiments, one strand of the stem portion of a double stranded RNA comprises a sequence that is similar or identical to the sequence of an intron, or a fragment thereof, in the pre-mRNA encoding a tissue abscission and/or inflorescence development time-modulating polypeptide, and the other strand of the stem portion comprises a sequence that is similar or identical to the sequence that is complementary to the sequence of the intron, or a fragment thereof, in the pre-mRNA.

The loop portion of a double stranded RNA can be from 3 nucleotides to 5,000 nucleotides, e.g., from 3 nucleotides to 25 nucleotides, from 15 nucleotides to 1,000 nucleotides, from 20 nucleotides to 500 nucleotides, or from 25 nucleotides to 200 nucleotides. The loop portion of the RNA can include an intron or a fragment thereof. A double stranded RNA can have zero, one, two, three, four, five, six, seven, eight, nine, ten, or more stem-loop structures.

A construct including a sequence that is operably linked to a regulatory region and a transcription termination sequence, and that is transcribed into an RNA that can form a double stranded RNA, is transformed into plants as described herein. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

Constructs containing regulatory regions operably linked to nucleic acid molecules in sense orientation can also be used to inhibit the expression of a gene. The transcription product can be similar or identical to the sense coding sequence, or a fragment thereof, of a tissue abscission and/or inflorescence development time-modulating polypeptide. The transcription product also can be unpolyadenylated, lack a 5′ cap structure, or contain an unspliceable intron. Methods of inhibiting gene expression using a full-length cDNA as well as a partial cDNA sequence are known in the art. See, e.g., U.S. Pat. No. 5,231,020.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can be a sequence that is identical or complementary to the sequence of an mRNA, the 3′ or 5′ untranslated region of an mRNA, or an intron in a pre-mRNA encoding a tissue abscission and/or inflorescence development time-modulating polypeptide, or a fragment of such sequences. In some embodiments, the sense or antisense sequence is identical or complementary to a sequence of the regulatory region that drives transcription of the gene encoding a tissue abscission and/or inflorescence development time-modulating polypeptide. In each case, the sense sequence is the sequence that is complementary to the antisense sequence.

The sense and antisense sequences can be any length greater than about 10 nucleotides (e.g., 12, 13, 14, 15, 16, 17, 18, 19, 20, 21, 22, 23, 24, 25, 26, 27, 28, 29, 30, or more nucleotides). For example, an antisense sequence can be 21 or 22 nucleotides in length. Typically, the sense and antisense sequences range in length from about 15 nucleotides to about 30 nucleotides, e.g., from about 18 nucleotides to about 28 nucleotides, or from about 21 nucleotides to about 25 nucleotides.

In some embodiments, an antisense sequence is a sequence complementary to an mRNA sequence, or a fragment thereof, encoding a tissue abscission and/or inflorescence development time-modulating polypeptide described herein. The sense sequence complementary to the antisense sequence can be a sequence present within the mRNA of the tissue abscission and/or inflorescence development time-modulating polypeptide. Typically, sense and antisense sequences are designed to correspond to a 15-30 nucleotide sequence of a target mRNA such that the level of that target mRNA is reduced.

In some embodiments, a construct containing a nucleic acid having at least one strand that is a template for more than one sense sequence (e.g., 2, 3, 4, 5, 6, 7, 8, 9, 10 or more sense sequences) can be used to inhibit the expression of a gene. Likewise, a construct containing a nucleic acid having at least one strand that is a template for more than one antisense sequence (e.g., 2, 3, 4, 5, 6, 7, 8, 9, 10 or more antisense sequences) can be used to inhibit the expression of a gene. For example, a construct can contain a nucleic acid having at least one strand that is a template for two sense sequences and two antisense sequences. The multiple sense sequences can be identical or different, and the multiple antisense sequences can be identical or different. For example, a construct can have a nucleic acid having one strand that is a template for two identical sense sequences and two identical antisense sequences that are complementary to the two identical sense sequences. Alternatively, an isolated nucleic acid can have one strand that is a template for (1) two identical sense sequences 20 nucleotides in length, (2) one antisense sequence that is complementary to the two identical sense sequences 20 nucleotides in length, (3) a sense sequence 30 nucleotides in length, and (4) three identical antisense sequences that are complementary to the sense sequence 30 nucleotides in length. The constructs provided herein can be designed to have any arrangement of sense and antisense sequences. For example, two identical sense sequences can be followed by two identical antisense sequences or can be positioned between two identical antisense sequences.

A nucleic acid having at least one strand that is a template for one or more sense and/or antisense sequences can be operably linked to a regulatory region to drive transcription of an RNA molecule containing the sense and/or antisense sequence(s). In addition, such a nucleic acid can be operably linked to a transcription terminator sequence, such as the terminator of the nopaline synthase (nos) gene. In some cases, two regulatory regions can direct transcription of two transcripts: one from the top strand, and one from the bottom strand. See, for example, Yan et al., Plant Physiol., 141:1508-1518 (2006). The two regulatory regions can be the same or different. The two transcripts can form double-stranded RNA molecules that induce degradation of the target RNA. In some cases, a nucleic acid can be positioned within a T-DNA or plant-derived transfer DNA (P-DNA) such that the left and right T-DNA border sequences, or the left and right border-like sequences of the P-DNA, flank or are on either side of the nucleic acid. See, US 2006/0265788. The nucleic acid sequence between the two regulatory regions can be from about 15 to about 300 nucleotides in length. In some embodiments, the nucleic acid sequence between the two regulatory regions is from about 15 to about 200 nucleotides in length, from about 15 to about 100 nucleotides in length, from about 15 to about 50 nucleotides in length, from about 18 to about 50 nucleotides in length, from about 18 to about 40 nucleotides in length, from about 18 to about 30 nucleotides in length, or from about 18 to about 25 nucleotides in length.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite, or an alkyl phosphotriester backbone.

### C. Constructs/Vectors

Recombinant constructs provided herein can be used to transform plants or plant cells in order to modulate tissue abscission levels and/or inflorescence development time. A recombinant nucleic acid construct can comprise a nucleic acid encoding a tissue abscission and/or inflorescence development time-modulating polypeptide as described herein, operably linked to a regulatory region suitable for expressing the tissue abscission and/or inflorescence development time-modulating polypeptide in the plant or cell. Thus, a nucleic acid can comprise a coding sequence that encodes any of the tissue abscission and/or inflorescence development time-modulating polypeptides as set forth in SEQ ID NOs: 3, 4, 6, 8, 10, 11, 12, 14, 16, 18, 19, 21, 22, 24, 25, 26, 27, 28, 31, 32, 34, 36, 38, 40, 42, 44, 46, 47, 49, 51, 53, 55, 57, 59, 61, 63, 65, 67, or 69. Examples of nucleic acids encoding tissue abscission and/or inflorescence development time-modulating polypeptides are set forth in SEQ ID NO: 1, 2, 5, 7, 9, 13, 15, 17, 20, 23, 29, 30, 33, 35, 37, 39, 41, 43, 45, 48, 50, 52, 54, 56, 58, 60, 62, 64, 66, or 68. The tissue abscission and/or inflorescence development time-modulating polypeptide encoded by a recombinant nucleic acid can be a native tissue abscission and/or inflorescence development time-modulating polypeptide, or can be heterologous to the cell. In some cases, the recombinant construct contains a nucleic acid that inhibits expression of a tissue abscission and/or inflorescence development time-modulating polypeptide, operably linked to a regulatory region. Examples of suitable regulatory regions are described in the section entitled “Regulatory Regions.”

Vectors containing recombinant nucleic acid constructs such as those described herein also are provided. Suitable vector backbones include, for example, those routinely used in the art such as plasmids, viruses, artificial chromosomes, BACs, YACs, or PACs. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., glyphosate, chlorsulfuron or phosphinothricin). In addition, an expression vector can include a tag sequence designed to facilitate manipulation or detection (e.g., purification or localization) of the expressed polypeptide. Tag sequences, such as luciferase, β-glucuronidase (GUS), green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

### D. Regulatory Regions

The choice of regulatory regions to be included in a recombinant construct depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence. Transcription of a nucleic acid can be modulated in a similar manner.

Some suitable regulatory regions initiate transcription only, or predominantly, in certain cell types. Methods for identifying and characterizing regulatory regions in plant genomic DNA are known, including, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of regulatory regions are described below. Some of the regulatory regions indicated below as well as additional regulatory regions are described in more detail in U.S. patent application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 11/408,791; 11/414,142; 10/950,321; 11/360,017; PCT/US05/011105; PCT/US05/23639; PCT/US05/034308; PCT/US05/034343; and PCT/US06/038236; PCT/US06/040572; and PCT/US07/62762.

For example, the sequences of regulatory regions p326, YP0144, YP0190, p13879, YP0050, p32449, 21876, YP0158, YP0214, YP0380, PT0848, PT0633, YP0128, YP0275, PT0660, PT0683, PT0758, PT0613, PT0672, PT0688, PT0837, YP0092, PT0676, PT0708, YP0396, YP0007, YP0111, YP0103, YP0028, YP0121, YP0008, YP0039, YP0115, YP0119, YP0120, YP0374, YP0101, YP0102, YP0110, YP0117, YP0137, YP0285, YP0212, YP0097, YP0107, YP0088, YP0143, YP0156, PT0650, PT0695, PT0723, PT0838, PT0879, PT0740, PT0535, PT0668, PT0886, PT0585, YP0381, YP0337, PT0710, YP0356, YP0385, YP0384, YP0286, YP0377, PD1367, PT0863, PT0829, PT0665, PT0678, YP0086, YP0188, YP0263, PT0743 and YP0096 are set forth in the sequence listing of PCT/US06/040572; the sequence of regulatory region PT0625 is set forth in the sequence listing of PCT/US05/034343; the sequences of regulatory regions PT0623, YP0388, YP0087, YP0093, YP0108, YP0022 and YP0080 are set forth in the sequence listing of U.S. patent application Ser. No. 11/172,703; the sequence of regulatory region PR0924 is set forth in the sequence listing of PCT/US07/62762; and the sequences of regulatory regions p530c10, pOsFIE2-2, pOsMEA, pOsYp102, and pOsYp285 are set forth in the sequence listing of PCT/US06/038236.

It will be appreciated that a regulatory region may meet criteria for one classification based on its activity in one plant species, and yet meet criteria for a different classification based on its activity in another plant species.

### i. Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326, YP0144, YP0190, p13879, YP0050, p32449, 21876, YP0158, YP0214, YP0380, PT0848, and PT0633 promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

### ii. Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128, YP0275, PT0625, PT0660, PT0683, and PT0758 promoters. Other root-preferential promoters include the PT0613, PT0672, PT0688, and PT0837 promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990), and the tobacco RD2 promoter.

### iii. Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1 (9):839-853 (1989)), the soybean trypsin inhibitor promoter (Riggs et al., Plant Cell, 1 (6):609-621 (1989)), the ACP promoter (Baerson et al., Plant Mol. Biol., 22 (2):255-267 (1993)), the stearoyl-ACP desaturase promoter (Slocombe et al., Plant Physiol., 104 (4):167-176 (1994)), the soybean α′ subunit of β-conglycinin promoter (Chen et al., Proc. Natl. Acad. Sci. USA, 83:8560-8564 (1986)), the oleosin promoter (Hong et al., Plant Mol. Biol., 34 (3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell Biol., 13:5829-5842 (1993)), the beta-amylase promoter, and the barley hordein promoter. Other maturing endosperm promoters include the YP0092, PT0676, and PT0708 promoters.

### iv. Ovary Tissue Promoters

Promoters that are active in ovary tissues such as the ovule wall and mesocarp can also be useful, e.g., a polygalacturonidase promoter, the banana TRX promoter, the melon actin promoter, YP0396, and PT0623. Examples of promoters that are active primarily in ovules include YP0007, YP0111, YP0092, YP0103, YP0028, YP0121, YP0008, YP0039, YP0115, YP0119, YP0120, and YP0374.

### v. Embryo Sac/Early Endosperm Promoters

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039, YP0101, YP0102, YP0110, YP0117, YP0119, YP0137, DME, YP0285, and YP0212. Other promoters that may be useful include the following rice promoters: p530c10, pOsFIE2-2, pOsMEA, pOsYp102, and pOsYp285.

### vi. Embryo Promoters

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable. Embryo-preferential promoters include the barley lipid transfer protein (Ltp1) promoter (Plant Cell Rep (2001) 20:647-654), YP0097, YP0107, YP0088, YP0143, YP0156, PT0650, PT0695, PT0723, PT0838, PT0879, and PT0740.

### vii. Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535, PT0668, PT0886, YP0144, YP0380 and PT0585.

### viii. Vascular Tissue Promoters

Examples of promoters that have high or preferential activity in vascular bundles include YP0087, YP0093, YP0108, YP0022, and YP0080. Other vascular tissue-preferential promoters include the glycine-rich cell wall protein GRP 1.8 promoter (Keller and Baumgartner, Plant Cell, 3 (10):1051-1061 (1991)), the Commelina yellow mottle virus (CoYMV) promoter (Medberry et al., Plant Cell, 4 (2):185-192 (1992)), and the rice tungro bacilliform virus (RTBV) promoter (Dai et al., Proc. Natl. Acad. Sci. USA, 101 (2):687-692 (2004)).

### ix. Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380, PT0848, YP0381, YP0337, PT0633, YP0374, PT0710, YP0356, YP0385, YP0396, YP0388, YP0384, PT0688, YP0286, YP0377, PD1367, and PD0901. Examples of nitrogen-inducible promoters include PT0863, PT0829, PT0665, and PT0886. Examples of shade-inducible promoters include PR0924 and PT0678. An example of a promoter induced by salt is rd29A (Kasuga et al. (1999) Nature Biotech 17: 287-291).

### x. Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

### xi. Stem Promoters

A stem promoter may be specific to one or more stem tissues or specific to stem and other plant parts. Stem promoters may have high or preferential activity in, for example, epidermis and cortex, vascular cambium, procambium, or xylem. Examples of stem promoters include YP0018 which is disclosed in US20060015970 and CryIA(b) and CryIA(c) (Braga et al. 2003, Journal of New Seeds 5:209-221).

### xii. Other Promoters

Other classes of promoters include, but are not limited to, shoot-preferential, callus-preferential, trichome cell-preferential, guard cell-preferential such as PT0678, tuber-preferential, parenchyma cell-preferential, and senescence-preferential promoters. Promoters designated YP0086, YP0188, YP0263, PT0758, PT0743, PT0829, YP0119, and YP0096, as described in the above-referenced patent applications, may also be useful.

### xiii. Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, for example, more than one regulatory region can be operably linked to the sequence of a polynucleotide encoding a tissue abscission and/or inflorescence development time-modulating polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### IV. Transgenic Plants and Plant Cells

### A. Transformation

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant provided the progeny inherits the transgene. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. A solid medium can be, for example, Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous tissue abscission and/or inflorescence development time-modulating polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

### B. Screening/Selection

A population of transgenic plants can be screened and/or selected for those members of the population that have a trait or phenotype conferred by expression of the transgene. For example, a population of progeny of a single transformation event can be screened for those plants having a desired level of expression of a tissue abscission and/or inflorescence development time-modulating polypeptide or nucleic acid. Physical and biochemical methods can be used to identify expression levels. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known. As an alternative, a population of plants comprising independent transformation events can be screened for those plants having a desired trait, such as a modulated level of tissue abscission and/or inflorescence development time. Selection and/or screening can be carried out over one or more generations, and/or in more than one geographic location. In some cases, transgenic plants can be grown and selected under conditions which induce a desired phenotype or are otherwise necessary to produce a desired phenotype in a transgenic plant. In addition, selection and/or screening can be applied during a particular developmental stage in which the phenotype is expected to be exhibited by the plant. Selection and/or screening can be carried out to choose those transgenic plants having a statistically significant difference in a tissue abscission level and/or inflorescence development time relative to a control plant that lacks the transgene. Selected or screened transgenic plants have an altered phenotype as compared to a corresponding control plant, as described in the “Transgenic Plant Phenotypes” section herein.

### C. Plant Species

The polynucleotides and vectors described herein can be used to transform a number of monocotyledonous and dicotyledonous plants and plant cell systems, including species from one of the following families: Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, or Vitaceae.

Suitable species may include members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

Suitable species include Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale (triticum—wheat X rye) and bamboo.

Suitable species also include Helianthus annuus (sunflower), Carthamus tinctorius (safflower), Jatropha curcas (jatropha), Ricinus communis (castor), Elaeis guineensis (palm), Linum usitatissimum (flax), and Brassica juncea.

Suitable species also include Beta vulgaris (sugarbeet), and Manihot esculenta (cassava)

Suitable species also include Lycopersicon esculentum (tomato), Lactuca sativa (lettuce), Musa paradisiaca (banana), Solanum tuberosum (potato), Brassica oleracea (broccoli, cauliflower, Brussels sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), and Solanum melongena (eggplant).

Suitable species also include Papaver somniferum (opium poppy), Papaver orientale, Taxus baccata, Taxus brevifolia, Artemisia annua, Cannabis sativa, Camptotheca acuminate, Catharanthus roseus, Vinca rosea, Cinchona officinalis, Colchicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, and Tanacetum parthenium.

Suitable species also include Parthenium argentatum (guayule), Hevea spp. (rubber), Mentha spicata (mint), Mentha piperita (mint), Bixa orellana, and Alstroemeria spp.

Suitable species also include Rosa spp. (rose), Dianthus caryophyllus (carnation), Petunia spp. (petunia) and Poinsettia pulcherrima (poinsettia).

Suitable species also include Nicotiana tabacum (tobacco), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass) and Phleum pratense (timothy).

In some embodiments, a suitable species can be a wild, weedy, or cultivated Pennisetum species such as, but not limited to, Pennisetum alopecuroides, Pennisetum arnhemicum, Pennisetum caffrum, Pennisetum clandestinum, Pennisetum divisum, Pennisetum glaucum, Pennisetum latifolium, Pennisetum macrostachyum, Pennisetum macrourum, Pennisetum orientale, Pennisetum pedicellatum, Pennisetum polystachion, Pennisetum polystachion ssp. Setosum, Pennisetum purpureum, Pennisetum setaceum, Pennisetum subangustum, Pennisetum typhoides, Pennisetum villosum, or hybrids thereof (e.g., Pennisetum purpureum×Pennisetum typhoidum).

In some embodiments, a suitable species can be a wild, weedy, or cultivated Miscanthus species and/or variety such as, but not limited to, Miscanthus×giganteus, Miscanthus sinensis, Miscanthus×ogiformis, Miscanthus floridulus, Miscanthus transmorrisonensis, Miscanthus oligostachyus, Miscanthus nepalensis, Miscanthus sacchariflorus, Miscanthus×giganteus ‘Amuri’, Miscanthus×giganteus ‘Nagara’, Miscanthus×giganteus ‘Illinois’, Miscanthus sinensis var. ‘Goliath’, Miscanthus sinensis var. ‘Roland’, Miscanthus sinensis var. ‘Africa’, Miscanthus sinensis var. ‘Fern Osten’, Miscanthus sinensis var. gracillimus, Miscanthus sinensis var. variegates, Miscanthus sinensis var. purpurascens, Miscanthus sinensis var. ‘Malepartus’, Miscanthus sacchariflorus var. ‘Robusta’, Miscanthus sinensis var. ‘Silberfedher’ (aka. Silver Feather), Miscanthus transmorrisonensis, Miscanthus condensatus, Miscanthus yakushimanum, Miscanthus var. ‘Alexander’, Miscanthus var. ‘Adagio’, Miscanthus var. ‘Autumn Light’, Miscanthus var. ‘Cabaret’, Miscanthus var. ‘Condensatus’, Miscanthus var. ‘Cosmopolitan’, Miscanthus var. ‘Dixieland’, Miscanthus var. ‘Gilded Tower’ (U.S. Pat. No. PP14,743), Miscanthus var. ‘Gold Bar’ (U.S. Pat. No. PP15,193), Miscanthus var. ‘Gracillimus’, Miscanthus var. ‘Graziella’, Miscanthus var. ‘Grosse Fontaine’, Miscanthus var. ‘Hinjo aka Little Nicky’™, Miscanthus var. ‘Juli’, Miscanthus var. ‘Kaskade’, Miscanthus var. ‘Kirk Alexander’, Miscanthus var. ‘Kleine Fontaine’, Miscanthus var. ‘Kleine Silberspinne’ (aka. ‘Little Silver Spider’), Miscanthus var. ‘Little Kitten’, Miscanthus var. ‘Little Zebra’ (U.S. Pat. No. PP13,008), Miscanthus var. ‘Lottum’, Miscanthus var. ‘Malepartus’, Miscanthus var. ‘Morning Light’, Miscanthus var. ‘Mysterious Maiden’ (U.S. Pat. No. PP16,176), Miscanthus var. ‘Nippon’, Miscanthus var. ‘November Sunset’, Miscanthus var. ‘Parachute’, Miscanthus var. ‘Positano’, Miscanthus var. ‘Puenktchen’ (aka ‘Little Dot’), Miscanthus var. ‘Rigoletto’, Miscanthus var. ‘Sarabande’, Miscanthus var. ‘Silberpfeil’ (aka. Silver Arrow), Miscanthus var. ‘Silverstripe’, Miscanthus var. ‘Super Stripe’ (U.S. Pat. No. PP18,161), Miscanthus var. ‘Strictus’, or Miscanthus var. ‘Zebrinus’.

In some embodiments, a suitable species can be a wild, weedy, or cultivated sorghum species and/or variety such as, but not limited to, Sorghum almum, Sorghum amplum, Sorghum angustum, Sorghum arundinaceum, Sorghum bicolor (such as bicolor, guinea, caudatum, kafir, and durra), Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideum, Sorghum sudanensese, Sorghum timorense, Sorghum trichocladum, Sorghum versicolor, Sorghum virgatum, Sorghum vulgare, or hybrids such as Sorghum×almum, Sorghum×sudangrass or Sorghum×drummondii.

Thus, the methods and compositions can be used over a broad range of plant species, including species from the dicot genera Brassica, Carthamus, Glycine, Gossypium, Helianthus, Jatropha, Parthenium, Populus, and Ricinus; and the monocot genera Elaeis, Festuca, Hordeum, Lolium, Oryza, Panicum, Pennisetum, Phleum, Poa, Saccharum, Secale, Sorghum, Triticosecale, Triticum, and Zea. In some embodiments, a plant is a member of the species Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet).

In certain embodiments, the polynucleotides and vectors described herein can be used to transform a number of monocotyledonous and dicotyledonous plants and plant cell systems, wherein such plants are hybrids of different species or varieties of a specific species (e.g., Saccharum sp.×Miscanthus sp., Sorghum sp.×Miscanthus sp., e.g., Panicum virgatum×Panicum amarum, Panicum virgatum×Panicum amarulum, and Pennisetum purpureum×Pennisetum typhoidum).

### D. Transgenic Plant Phenotypes

In some embodiments, a plant in which expression of a tissue abscission and/or inflorescence development time-modulating polypeptide is modulated can have delayed abscission and/or modulated inflorescence development. For example, a tissue abscission and/or inflorescence development time-modulating polypeptide described herein can be expressed in a transgenic plant, resulting in increased delayed abscission. The abscission can be delayed by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the abscission level in a corresponding control plant that does not express the transgene. In some embodiments, a plant in which expression of a tissue abscission and/or inflorescence development time-modulating polypeptide is modulated can have decreased levels of abscission. The abscission level can be decreased by at least 2 percent, e.g., 2, 3, 4, 5, 10, 15, 20, 25, 30, 35, or more than 35 percent, as compared to the abscission level in a corresponding control plant that does not express the transgene.

Increases in abscission in such plants can provide improved agronomic traits in such as increase biomass or more uniform flowering and seed set. Decreases in abscission in such plants can be useful in situations where flowers or other plant organs that abscise are not the primary plant part that is harvested for human or animal consumption.

In some embodiments, a plant in which expression of a tissue abscission and/or inflorescence development time-modulating polypeptide is modulated can have increased or decreased levels of abscission in one or more tissues, e.g., petal tissues, or leaf tissues. For example, the abscission level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the abscission level in a corresponding control plant that does not express the transgene. In some embodiments, a plant in which expression of a tissue abscission and/or inflorescence development time-modulating polypeptide is modulated can have decreased levels of abscission in one or more tissues. The abscission level can be decreased by at least 2 percent, e.g., 2, 3, 4, 5, 10, 15, 20, 25, 30, 35, or more than 35 percent, as compared to the abscission level in a corresponding control plant that does not express the transgene.

Typically, a difference in the amount of abscission in a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the amount of abscission is statistically significant at p<0.01, p<0.005, or p<0.001. A statistically significant difference in, for example, the amount of abscission in a transgenic plant compared to the amount in cells of a control plant indicates that the recombinant nucleic acid present in the transgenic plant results in altered abscission levels.

The phenotype of a transgenic plant is evaluated relative to a control plant. A plant is said “not to express” a polypeptide when the plant exhibits less than 10%, e.g., less than 9%, 8%, 7%, 6%, 5%, 4%, 3%, 2%, 1%, 0.5%, 0.1%, 0.01%, or 0.001%, of the amount of polypeptide or mRNA encoding the polypeptide exhibited by the plant of interest. Expression can be evaluated using methods including, for example, RT-PCR, Northern blots, 51 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

### V. Plant Breeding

Genetic polymorphisms are discrete allelic sequence differences in a population. Typically, an allele that is present at 1% or greater is considered to be a genetic polymorphism. The discovery that polypeptides disclosed herein can modulate tissue abscission and/or inflorescence development time is useful in plant breeding, because genetic polymorphisms exhibiting a degree of linkage with loci for such polypeptides are more likely to be correlated with variation in a tissue abscission and/or inflorescence development time trait. For example, genetic polymorphisms linked to the loci for such polypeptides are more likely to be useful in marker-assisted breeding programs to create lines having a desired modulation in the tissue abscission and/or inflorescence development time trait.

Thus, one aspect of the invention includes methods of identifying whether one or more genetic polymorphisms are associated with variation in a tissue abscission and/or inflorescence development time trait. Such methods involve determining whether genetic polymorphisms in a given population exhibit linkage with the locus for one of the polypeptides depicted in FIGS. 1 and 2 and/or a functional homolog thereof, such as, but not limited to those identified in the Sequence Listing of this application. The correlation is measured between variation in the tissue abscission and/or inflorescence development time trait in plants of the population and the presence of the genetic polymorphism(s) in plants of the population, thereby identifying whether or not the genetic polymorphism(s) are associated with variation for the trait. If the presence of a particular allele is statistically significantly correlated with a desired modulation in the tissue abscission and/or inflorescence development time trait, the allele is associated with variation for the trait and is useful as a marker for the trait. If, on the other hand, the presence of a particular allele is not significantly correlated with the desired modulation, the allele is not associated with variation for the trait and is not useful as a marker.

Such methods are applicable to populations containing the naturally occurring endogenous polypeptide rather than an exogenous nucleic acid encoding the polypeptide, i.e., populations that are not transgenic for the exogenous nucleic acid. It will be appreciated, however, that populations suitable for use in the methods may contain a transgene for another, different trait, e.g., herbicide resistance.

Genetic polymorphisms that are useful in such methods include simple sequence repeats (SSRs, or microsatellites), rapid amplification of polymorphic DNA (RAPDs), single nucleotide polymorphisms (SNPs), amplified fragment length polymorphisms (AFLPs) and restriction fragment length polymorphisms (RFLPs). SSR polymorphisms can be identified, for example, by making sequence specific probes and amplifying template DNA from individuals in the population of interest by PCR. For example, PCR techniques can be used to enzymatically amplify a genetic marker associated with a nucleotide sequence conferring a specific trait (e.g., nucleotide sequences described herein). PCR can be used to amplify specific sequences from DNA as well as RNA, including sequences from total genomic DNA or total cellular RNA. When using RNA as a source of template, reverse transcriptase can be used to synthesize complementary DNA (cDNA) strands. Various PCR methods are described, for example, in PCR Primer: A Laboratory Manual, Dieffenbach and Dveksler, eds., Cold Spring Harbor Laboratory Press, 1995.

Generally, sequence information from polynucleotides flanking the region of interest or beyond is employed to design oligonucleotide primers that are identical or similar in sequence to opposite strands of the template to be amplified. Primers are typically 14 to 40 nucleotides in length, but can range from 10 nucleotides to hundreds of nucleotides in length. Template and amplified DNA is repeatedly denatured at a high temperature to separate the double strand, then cooled to allow annealing of primers and the extension of nucleotide sequences through the microsatellite, resulting in sufficient DNA for detection of PCR products. If the probes flank an SSR in the population, PCR products of different sizes will be produced. See, e.g., U.S. Pat. No. 5,766,847.

PCR products can be qualitative or quantitatively analyzed using several techniques. For example, PCR products can be stained with a fluorescent molecule (e.g., PicoGreen® or OliGreen®) and detected in solution using spectrophotometry or capillary electrophoresis. In some cases, PCR products can be separated in a gel matrix (e.g., agarose or polyacrylamide) by electrophoresis, and size-fractionated bands comprising PCR products can be visualized using nucleic acid stains. Suitable stains can fluoresce under UV light (e.g., Ethidium bromide, GR Safe, SYBR® Green, or SYBR® Gold). The results can be visualized via transillumination or epi-illumination, and an image of the fluorescent pattern can be acquired using a camera or scanner, for example. The image can be processed and analyzed using specialized software (e.g., ImageJ) to measure and compare the intensity of a band of interest against a standard loaded on the same gel.

Alternatively, SSR polymorphisms can be identified by using PCR product(s) as a probe against Southern blots from different individuals in the population. See, U. H. Refseth et al., (1997) Electrophoresis 18: 1519. Briefly, PCR products are separated by length through gel electrophoresis and transferred to a membrane. SSR-specific DNA probes, such as oligonucleotides labeled with radioactive, fluorescent, or chromogenic molecules, are applied to the membrane and hybridize to bound PCR products with a complementary nucleotide sequence. The pattern of hybridization can be visualized by autoradiography or by development of color on the membrane, for example.

In some cases, PCR products can be quantified using a real-time thermocycler detection system. For example, Quantitative real-time PCR can use a fluorescent dye that forms a DNA-dye-complex (e.g., SYBR® Green), or a fluorophore-containing DNA probe, such as single-stranded oligonucleotides covalently bound to a fluorescent reporter or fluorophore (e.g. 6-carboxyfluorescein or tetrachlorofluorescin) and quencher (e.g., tetramethylrhodamine or dihydrocyclopyrroloindole tripeptide minor groove binder). The fluorescent signal allows detection of the amplified product in real time, thereby indicating the presence of a sequence of interest, and allowing quantification of the copy number of a sequence of interest in cellular DNA or expression level of a sequence of interest from cellular mRNA.

The identification of RFLPs is discussed, for example, in Alonso-Blanco et al. (Methods in Molecular Biology, vol.82, “Arabidopsis Protocols”, pp. 137-146, J. M.

Martinez-Zapater and J. Salinas, eds., c. 1998 by Humana Press, Totowa, N.J.); Burr (“Mapping Genes with Recombinant Inbreds”, pp. 249-254, in Freeling, M. and V. Walbot (Ed.), The Maize Handbook, c. 1994 by Springer-Verlag New York, Inc.: New York, N.Y., USA; Berlin Germany; Burr et al. Genetics (1998) 118: 519; and Gardiner, J. et al., (1993) Genetics 134: 917). For example, to produce a RFLP library enriched with single- or low-copy expressed sequences, total DNA can be digested with a methylation-sensitive enzyme (e.g., PstI). The digested DNA can be separated by size on a preparative gel. Polynucleotide fragments (500 to 2000 bp) can be excised, eluted and cloned into a plasmid vector (e.g., pUC18). Southern blots of plasmid digests can be probed with total sheared DNA to select clones that hybridize to single- and low-copy sequences. Additional restriction endonucleases can be tested to increase the number of polymorphisms detected.

The identification of AFLPs is discussed, for example, in EP 0 534 858 and U.S. Pat. No. Pat. 5,878,215. In general, total cellular DNA is digested with one or more restriction enzymes. Restriction halfsite-specific adapters are ligated to all restriction fragments and the fragments are selectively amplified with two PCR primers that have corresponding adaptor and restriction site specific sequences. The PCR products can be visualized after size-fractionation, as described above.

In some embodiments, the methods are directed to breeding a plant line. Such methods use genetic polymorphisms identified as described above in a marker assisted breeding program to facilitate the development of lines that have a desired alteration in the tissue abscission and/or inflorescence development time trait. Once a suitable genetic polymorphism is identified as being associated with variation for the trait, one or more individual plants are identified that possess the polymorphic allele correlated with the desired variation. Those plants are then used in a breeding program to combine the polymorphic allele with a plurality of other alleles at other loci that are correlated with the desired variation. Techniques suitable for use in a plant breeding program are known in the art and include, without limitation, backcrossing, mass selection, pedigree breeding, bulk selection, crossing to another population and recurrent selection. These techniques can be used alone or in combination with one or more other techniques in a breeding program. Thus, each identified plants is selfed or crossed a different plant to produce seed which is then germinated to form progeny plants. At least one such progeny plant is then selfed or crossed with a different plant to form a subsequent progeny generation. The breeding program can repeat the steps of selfing or outcrossing for an additional 0 to 5 generations as appropriate in order to achieve the desired uniformity and stability in the resulting plant line, which retains the polymorphic allele. In most breeding programs, analysis for the particular polymorphic allele will be carried out in each generation, although analysis can be carried out in alternate generations if desired.

In some cases, selection for other useful traits is also carried out, e.g., selection for fungal resistance or bacterial resistance. Selection for such other traits can be carried out before, during or after identification of individual plants that possess the desired polymorphic allele.

### VI. Articles of Manufacture

Transgenic plants provided herein have various uses in the agricultural and energy production industries. For example, transgenic plants described herein can be used to make animal feed and food products. Such plants, however, are often particularly useful as a feedstock for energy production.

Transgenic plants described herein often produce higher yields of grain and/or biomass per hectare because less energy is devoted to flowers or plant organs that abscise, relative to control plants that lack the exogenous nucleic acid. In some embodiments, such transgenic plants provide equivalent or even increased yields of biomass per hectare relative to control plants when grown under conditions of reduced inputs such as fertilizer and/or water. Thus, such transgenic plants can be used to provide stability at a lower input cost and/or under environmentally stressful conditions such as drought. In some embodiments, plants described herein have a composition that permits more efficient processing into free sugars, and subsequently ethanol, for energy production. In some embodiments, such plants provide higher yields of ethanol, butanol, dimethyl ether, other biofuel molecules, and/or sugar-derived co-products per kilogram of plant material, relative to control plants. Such processing efficiencies are believed to be derived from the lignin, sugar, cellulose, and hemicellulose composition of the plant material. By providing higher yields at an equivalent or even decreased cost of production, the transgenic plants described herein improve profitability for farmers and processors as well as decrease costs to consumers. In other embodiments, the transgenic plants of the invention having increase abscission can be used for confinement purposes by, for example, expressing an antisense version of the delayed abscission genes one could increase abscission of flowering organs so that seeds are not given a chance to develop and the plants are more sterile.

Seeds from transgenic plants described herein can be conditioned and bagged in packaging material by means known in the art to form an article of manufacture. Packaging material such as paper and cloth are well known in the art. A package of seed can have a label, e.g., a tag or label secured to the packaging material, a label printed on the packaging material, or a label inserted within the package, that describes the nature of the seeds therein.

The invention will be further described in the following examples, which do not limit the scope of the invention described in the claims.

### VII. EXAMPLES

### Example 1

### Transgenic Arabidopsis Plants

The following symbols are used in the Examples with respect to Arabidopsis transformation: T₁: first generation transformant; T₂: second generation, progeny of self-pollinated T₁ plants; T₃: third generation, progeny of self-pollinated T₂ plants; T₄: fourth generation, progeny of self-pollinated T₃ plants. Independent transformations are referred to as events.

The following is a list of nucleic acids that were isolated from Arabidopsis thaliana plants, CeresClone:32430 and CeresClone:1678.

Each isolated nucleic acid described above was cloned into a Ti plasmid vector, CRS338, containing a phosphinothricin acetyltransferase gene which confers Finale™ resistance to transformed plants. Constructs were made using CRS338 that contained CeresClone:32430 or CeresClone:1678, each operably linked to a 35S promoter. Wild-type Arabidopsis thaliana ecotype Wassilewskija (Ws) plants were transformed separately with each construct. The transformations were performed essentially as described in Bechtold et al., C.R. Acad. Sci. Paris, 316:1194-1199 (1993).

Transgenic Arabidopsis lines containing CeresClone:32430, or CeresClone:1678 were designated ME05885 or ME03564, respectively. The presence of each vector containing a nucleic acid described above in the respective transgenic Arabidopsis line transformed with the vector was confirmed by Finale™ resistance, PCR amplification from green leaf tissue extract, and/or sequencing of PCR products. As controls, wild-type Arabidopsis ecotype Ws plants and non-transgenic segregants

### Example 2

### A validated Assay for Analyzing Transgenic Plants for Delayed Petal Abscission

Soil is prepared by mixing 60% autoclaved Sunshine Mix #5™ with 40% vermiculite in 24L increments. Three Tbsp of Marathon™ are added per 24 L of soil prepared. Soil is thoroughly mixed using an automatic soil mixer. The soil is turned for at least 10 minutes so that soil lumps are completely removed. The resulting soil mixture will henceforth be referred to as refined soil.

For each candidate×generation×event to be tested, 24 pots (8.0 cm×8.0 cm×8.0 cm) are prepared as follows. Pots are placed in one tray with holes on the bottom, and a no-hole-ridged bottom tray is placed underneath for watering. Pots are loosely filled with dry refined soil mix to the brim. Soil is allowed to settle with a quick shake and excess soil is removed from the top using the edge of a ruler. Using this methodology, each pot holds approximately 65 g of dry refined soil.

Four L of filtered water are added to the bottom of each no-hole-ridge tray and soil is allowed to wet for 2 hours prior to use (or overnight). This allows the soil to completely settle while wetting. Some water will remain in the bottom of the flat. Filtered water is sprinkled onto the surface of soil, but over-spraying is avoided to prevent draining soil away from the surface.

Seed sowing and stratification begin by labeling each pot with an identifier containing a barcode and event identifier. Five seeds are planted per pot and 24 pots for each candidate×generation×event. For each experiment (usually two events and one generation), 6 pots are planted of wild-type Ws. The two events and the control are evenly distributed among three flats such that 8 plants of each event and 2 Ws plants are placed in each flat. They are sown in three flats according to a Latin squares design modified to incorporate controls.

The flat containing the pots is covered with a dome and transferred to the dark at 4° C. for 3 to 7 days. After stratification, excess water is dumped off and the flats are transferred to a growth chamber (16:8 hour light:dark cycle; 150 uEinsteins; 70% relative humidity; 22° C.).

All the flats are treated in the same manner at each step. Humidity domes are removed after 5 days in the growth chamber or when cotyledons are fully expanded. Meanwhile, pots are watered with 2.5 L of half strength Hoagland's solution. The soil is soaked for two hours and then excess Hoagland's solution is dumped. On the 7th to 10th day after transfer to the growth chamber, seedlings are weeded such that only one seedling remained in each pot. Generally, the flats are watered alternately with 2.5 L of half strength Hoagland's solution or filtered water every 5 days. Excess solution is removed after each watering. The soil is not allowed to completely dry out and no water is allowed to remain in the flats 12 hours after watering.

Transgenic and non-transgenic segregants are identified as follows. Five days post-bolting, a portion of a cauline leaf from each plant including the control is harvested for Finale™ resistant:sensitive analysis. Finale™ resistance can be determined conventionally by spraying seedlings and waiting several days for symptoms to appear, or it can be determined shortly after spraying by measuring the photosynthetic efficiency with a CF Fluorescent Imager. In this protocol, the photosynthetic efficiency measurement is used.

The primary inflorescence of each plant is marked. The primary inflorescence is gently tied onto Hyacinth stakes when each plant reaches approximately 15 cm.

The date when the first flower opened is recorded for each plant. The number of flowers with turgid petals on the primary inflorescence 12 days after the first flower opens is also recorded. For statistical analysis, a T-test is used to determine whether the transgenic plants have significantly more flowers with turgid petals than the non-transgenic segregants.

### Example 3

### Results for ME03564 (SEQ ID NO:3) (Clone 1678; cDNA ID 23364915; At3g54200) Events

Ectopic expression of Clone 1678 under the control of the 35S promoter results in a delayed petal abscission compared to controls. Clone 1678 was identified as comprising a gene with unknown function. ME03564 was identified as an ethylene-insensitive line from a superpool screen. The transgene sequence was obtained for 9 candidate plants. Seven candidate sequences BLASTed to ME03564. T2 and T3 seed from 2 events of ME03564 containing 35S::clone 1678 was analyzed for delayed petal abscission using the assay described in Example 2. The Ti plasmid vector used for this construct, CRS 338, contains the Ceres-constructed, plant selectable marker gene phosphinothricin acetyltransferase (PAT) which confers Basta resistance to transformed plants.

Two events of ME03564 showed significantly delayed petal abscission in both generations. The number of flowers with turgid petals on the 10th day after bolting was used to represent the petal number. The number of flowers with petals of the transgenic plants within an event in one generation was compared with that of non-transgenic segregants pooled across both events in both generations. In comparison with the non-transgenic segregants and the external wild-type Ws controls, both events have significantly more flowers with petals in both generations at p=0.05, using a one-tailed t-test assuming unequal variance (Table 1).

TABLE 1
T-test comparison of the number of flowers with petals between transgenic plants and
pooled non-transgenic segregants.

| | | | Transgenic | | | Non-transgenic | | | |
|---|---|---|---|---|---|---|---|---|---|
| | | | Number of Flowers | | | Number of Flowers | | | |
| Line | Events | Generation | with Petals | SE | n | with Petals | SE | n | p-value |
| ME03564 | ME03564-02 | T2 | 17.83 | 0.44 | 23 | 15.38 | 0.30 | 26 | 1.74E−05 |
| | | T3 | 17.17 | 0.53 | 24 | 15.38 | 0.30 | 26 | 2.70E−03 |
| | ME03564-03 | T2 | 17.58 | 0.48 | 12 | 15.38 | 0.30 | 26 | 2.35E−04 |
| | | T3 | 17.80 | 0.49 | 10 | 15.38 | 0.30 | 26 | 9.40E−05 |

Segregation ratios for Finale ™ resistance were recorded. Events-02 and -03 segregated 15:1 and 3:1 (R:S) for Finale ™ resistance in the T₂ generation, respectively.

There was no noticeable difference in morphological appearance in the majority of the ten T1 plants compared to the controls.

Neither event exhibited any statistically relevant negative phenotypes. Plants from Events −02 and −03 which are hemizygous or homozygous for Clone 1678 do not show any significant negative phenotypes under standard growth conditions. The physical appearances of T₁, T₂, and T₃ plants were similar to those of corresponding control plants apart from delayed petal abscission. There were no observable or statistically significant differences between T₂ or T₃ plants from events −02 and −03 of ME03564 and control plants in germination, onset of flowering, rosette area, fertility (silique number and seed fill), and general morphology/architecture.

### Example 4

### Results for ME05885 (SEO ID NO: 31) (clone 32430) Events

Ectopic expression of clone 32430 under the control of the 35S promoter results in delayed petal abscission compared to controls. T3 and T4 seed from two events of

ME05885 containing clone 32430 was analyzed for delayed petal abscission using the assay described in Example 2. Clone 32430 was identified as a gene with unknown function. ME05885 was identified as an ethylene-insensitive line from a superpool screen. Transgene sequence was obtained for 7 candidate plants. One candidate sequence BLASTed to ME05885.

Two events of ME05885 showed significantly delayed petal abscission in both generations. The two events of ME05885 (−03 and −04) were chosen to be analyzed in an abscission assay as described in Example 2 in both the T3 and the T4 generations. In this assay, the number of flowers with turgid petals on the 10th day post-bolting for T3 generation and 12th day post-bolting for T4 generation was recorded. The number of flowers with petals of the transgenic plants within an event in one generation was compared to that of non-transgenic segregants pooled across both events in the same generation. In comparison with the non-transgenic segregants and the external wild-type Ws controls, both events had significantly more flowers with petals in both generations at p=0.05, using a one-tailed t-test assuming unequal variance (Table 2).

TABLE 2
t-test comparison of the number of flowers with petals between transgenic plants and
pooled non-transgenic segregants.

| | | | Transgenic | | | Non-transgenic | | | |
|---|---|---|---|---|---|---|---|---|---|
| | | | Numbr of Flowers | | | Number of Flowers | | | |
| Line | Events | Generation | with Petals | SE | N | with Petals | SE | N | p-value |
| ME05885 | ME05885-03 | T3 | 15.33 | 0.30 | 15 | 13.82 | 0.54 | 17 | 1.02E−02 |
| | | T4 | 16.50 | 0.79 | 10 | 14.65 | 0.19 | 23 | 1.53E−02 |
| | ME05885-04 | T3 | 16.50 | 0.55 | 16 | 13.82 | 0.54 | 17 | 7.40E−04 |
| | | T4 | 18.17 | 0.46 | 12 | 14.65 | 0.19 | 23 | 2.20E−08 |

Events -03 and -04 both segregated 3:1 (R:S) for Finale ™ resistance in the T₂ generation.

The physical appearances of T1 plants were similar to those of corresponding control plants. Neither event exhibited any statistically relevant negative phenotypes compared to the empty vector control SR00559. There were no observable or statistically significant differences between T3 and T4 plants from events −03 and −04 of ME05885 and control plants in germination, onset of flowering, rosette area, and fertility. With respect to general morphology/architecture, the siliques of some transgenic plants are slightly shorter than the controls. No other morphology/architecture defects were observed.

### Example 5

### Determination of Functional Homologs by Reciprocal BLAST

A candidate sequence was considered a functional homolog of a reference sequence if the candidate and reference sequences encoded proteins having a similar function and/or activity. A process known as Reciprocal BLAST (Rivera et al., Proc. Natl. Acad. Sci. USA, 95:6239-6244 (1998)) was used to identify potential functional homolog sequences from databases consisting of all available public and proprietary peptide sequences, including NR from NCBI and peptide translations from Ceres clones.

Before starting a Reciprocal BLAST process, a specific reference polypeptide was searched against all peptides from its source species using BLAST in order to identify polypeptides having BLAST sequence identity of 80% or greater to the reference polypeptide and an alignment length of 85% or greater along the shorter sequence in the alignment. The reference polypeptide and any of the aforementioned identified polypeptides were designated as a cluster.

The BLASTP version 2.0 program from Washington University at Saint Louis, Missouri, USA was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog sequence with a specific reference polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a reference polypeptide sequence, “polypeptide A,” from source species SA was BLASTed against all protein sequences from a species of interest. Top hits were determined using an E-value cutoff of 10⁻⁵ and a sequence identity cutoff of 35%. Among the top hits, the sequence having the lowest E-value was designated as the best hit, and considered a potential functional homolog or ortholog. Any other top hit that had a sequence identity of 80% or greater to the best hit or to the original reference polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog.

Functional homologs were identified by manual inspection of potential functional homolog sequences. Representative functional homologs for SEQ ID NO: 31 and SEQ ID NO: 3 are shown in FIGS. 1-2, respectively. Additional exemplary homologs are correlated to certain Figures in the Sequence Listing.

### Example 6

### Determination of Functional Homologs by Hidden Markov Models

Hidden Markov Models (HMMs) were generated by the program HMMER 2.3.2. To generate each HMM, the default HMMER 2.3.2 program parameters, configured for glocal alignments, were used.

An HMM was generated using the sequences shown in FIG. 1 as input. These sequences were fitted to the model and a representative HMM bit score for each sequence is shown in the Sequence Listing. Additional sequences were fitted to the model, and representative HMM bit scores for any such additional sequences are shown in the Sequence Listing. The results indicate that these additional sequences are functional homologs of SEQ ID NO: 31.

The procedure above was repeated and an HMM was generated for the group of sequences shown in FIG. 2 using the sequences shown in the Figure as input for that HMM. A representative bit score for each sequence is shown in the Sequence Listing. Additional sequences were fitted to certain HMMs, and representative HMM bit scores for such additional sequences are shown in the Sequence Listing. The results indicate that these additional sequences are functional homologs of the sequences used to generate that HMM.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A plant cell comprising an exogenous nucleic acid, wherein said exogenous nucleic acid comprises a regulatory region operably linked to a nucleotide sequence encoding a polypeptide, wherein said polypeptide has an HMM bit score greater than about 115 based on the amino acid sequences depicted in FIG. 1 or FIG. 2.

2. A plant cell comprising an exogenous nucleic acid, wherein said exogenous nucleic acid comprises a regulatory region operably linked to a nucleotide sequence encoding a polypeptide having 80 percent or greater sequence identity to an amino acid sequence selected from the group consisting of SEQ ID NO: 3, 4, 6, 8, 10, 11, 12, 14, 16, 18, 19, 21, 22, 24, 25, 26, 27, 28, 31, 32, 34, 36, 38, 40, 42, 44, 46, 47, 49, 51, 53, 55, 57, 59, 61, 63, 65, 67, and 69.

3. A plant cell comprising an exogenous nucleic acid, wherein said exogenous nucleic acid comprises a regulatory region operably linked to a nucleotide sequence having 80 percent or greater sequence identity to a nucleotide sequence selected from the group consisting of SEQ ID NO: 1, 2, 5, 7, 9, 13, 15, 17, 20, 23, 29, 30, 33, 35, 37, 39, 41, 43, 45, 48, 50, 52, 54, 56, 58, 60, 62, 64, 66, and 68, or a fragment thereof.

4. A transgenic plant comprising the plant cell of claim 1.

5. The transgenic plant of claim 4, wherein said plant is a member of a species selected from the group consisting of Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), and Pennisetum glaucum (pearl millet).

6. A transgenic plant comprising the plant cell of claim 4, wherein said polypeptide is selected from the group consisting of SEQ ID NO:3 and 31.

7. A seed product comprising embryonic tissue from a transgenic plant according to claim 4, wherein said seed product contains said exogenous nucleic acid.

8. An isolated nucleic acid comprising a nucleotide sequence having 85% or greater sequence identity to the nucleotide sequence set forth in SEQ ID NO: 35, 48, 7, 39, 13, 52, 62, 43, 17, 33, 5, 20, or 23.

9. An isolated nucleic acid comprising a nucleotide sequence encoding a polypeptide having 80% or greater sequence identity to the amino acid sequence set forth in SEQ ID NO: 36, 49, 8, 40, 14, 53, 63, 44, 18, 34, 6, 21, or 24.

10. A transgenic plant comprising the plant cell of claim 2.

11. The transgenic plant of claim 10, wherein said plant is a member of a species selected from the group consisting of Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), and Pennisetum glaucum (pearl millet).

12. A transgenic plant comprising the plant cell of claim 10, wherein said polypeptide is selected from the group consisting of SEQ ID NO:3 and 31.

13. A seed product comprising embryonic tissue from a transgenic plant according to claim 10, wherein said seed product contains said exogenous nucleic acid.

14. A transgenic plant comprising the plant cell of claim 3.

15. The transgenic plant of claim 14, wherein said plant is a member of a species selected from the group consisting of Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), and Pennisetum glaucum (pearl millet).

16. A transgenic plant comprising the plant cell of claim 14, wherein said polypeptide is selected from the group consisting of SEQ ID NO:3 and 31.

17. A seed product comprising embryonic tissue from a transgenic plant according to claim 14, wherein said seed product contains said exogenous nucleic acid.

18. A method of modulating the level of abscission in a plant comprising introducing into a plant cell an exogenous nucleic acid, wherein said exogenous nucleic acid comprises a regulatory region operably linked to a nucleotide sequence according to claim 8.

19. A method of modulating the level of abscission in a plant comprising introducing into a plant cell an exogenous nucleic acid, wherein said exogenous nucleic acid comprises a regulatory region operably linked to a nucleotide sequence according to claim 9.

20. The method according to claim 19, wherein said plant cell is from a plant that is a member of a species selected from the group consisting of Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), and Pennisetum glaucum (pearl millet).


SEQUENCE LISTING (69 sequences)

SEQ ID NO: 1 - DNA, 893 bp, Arabidopsis thaliana
  misc_feature: Expected sequence
aaaccaaaacccctgagaaactcaacagcgagagaaccacaaagcgtaacaaagagagtaataactatgagtgatttttcaatcaaacccgatgataaaaaggaagaggagaagccagcaacagccatgttaccaccacccaaaccaaacgcttcttccatggaaacgcaatctgctaacaccggaaccgctaaaaaactgcggcggaaacgcaactgcaaaatctgtatctgtttcactatccttctcattctcctaatcgccatagttatcgtcatcttagctttcactctcttcaaaccaaaacgcccaactactacaatcgattccgtcaccgttgatcgtctccaagcttccgtcaatcctctccttctcaaagtcctcctaaacctaacgctcaacgtcgatctctctctcaaaaaccctaaccgcatcgggtttagctacgattcgtcgtcggcgttgttgaattacagaggccaagtgatcggtgaagctcctcttccggctaaccgaatcgcggcgcggaaaacggtgccgttgaatataacgttgacgctaatggcggatcggttactctctgaaacgcagcttttatctgacgtcatggctggcgtcattccgcttaatacttttgttaaagtcactggtaaagtaaccgttcttaaaatctttaagattaaagtccaatcgtcttcttcgtgtgatctcagtatctctgtttcgaatcgtaatgttacgagtcaacactgtaagtattcgactaagttataatcagatctgtagattctaccacactttgttcttgttttgttttttggtatttgaattggtttgtatttaatctctatgtatgttttatttactttaatgaacagagatttgattttgtt

SEQ ID NO: 2 - DNA, 893 bp, Arabidopsis thaliana
  misc_feature: Inplanta sequence
aaaccaaaacccctgagaaactcaacagcgagagaaccacaaagcgtaacaaagagagtaataactatgagtgatttttcaatcaaacccgatgataaaaaggaagaggagaagccagcaacagccatgttaccaccacccaaaccaaacgcttcttccatggaaacgcaatctgctaacaccggaaccgctaaaaaactgcggcggaaacgcaactgcaaaatctgtatctgtttcactatccttctcattctcctaatcgccatagttatcgtcatcttagctttcactctcttcaaaccaaaacgcccaactactacaatcgattccgtcaccgttgatcgtctccaagcttccgtcaatcctctccttctcaaagtcctcctaaacctaacgctcaacgtcgatctctctctcaaaaaccctaaccgcatcgggtttagctacgattcgtcgtcggcgttgttgaattacagaggccaagtgatcggtgaagctcctcttccggctaaccgaatcgcggcgcggaaaacggtgtcgttgaatataacgttgacgctaatggcggatcggttactctctgaaacgcagcttttatctgacgtcatggctggcgtcattccgcttaatacttttgttaaagtcactggtaaagtaaccgttcttaaaatctttaagattaaagtccaatcgtcttcttcgtgtgatctcagtatctctgtttcgaatcgtaatgttacgagtcaacactgtaagtattcgactaagttataatcagatctgtagattctaccacactttgttcttgttttgttttttggtatttgaattggtttgtatttaatctctatgtatgttttatttactttaatgaacagagatttgattttgtt

SEQ ID NO: 3 - protein, 235 aa, Arabidopsis thaliana
  misc_feature: Ceres CLONE ID no. 1678
MSDFSIKPDDKKEEEKPATAMLPPPKPNASSMETQSANTGTAKKLRRKRNCKICICFTILLILLIAIVIVILAFTLFKPKRPTTTIDSVTVDRLQASVNPLLLKVLLNLTLNVDLSLKNPNRIGFSYDSSSALLNYRGQVIGEAPLPANRIAARKTVPLNITLTLMADRLLSETQLLSDVMAGVIPLNTFVKVTGKVTVLKIFKIKVQSSSSCDLSISVSNRNVTSQHCKYSTKL

SEQ ID NO: 4 - protein, 227 aa, Picea sitchensis
  misc_feature: Public GI ID no. 116782452
MAPASTSRSNPVPFQEPYSYGYPANAYYVVLPPAPHLHSRRRSRRLLAYAAALVVSLGLFLGAAFYIFWPTQPVIQVIGVNFNTINFQAAPQPGSVVPRLFMNISMDMALKVNNKDYFSLEYDSLDVGLGYRGRRIGVVESEGGRLPARHTAYVNSTLELDGVEIFHDTVYLLEDLVRKELPLDTVAEFNGSVRVLLVKVPLKKVVSCEVVVNPDNQTILSQDCGLV

SEQ ID NO: 5 - DNA, 1011 bp, Gossypium hirsutum
  misc_feature: Encodes the peptide sequence at SEQ ID NO. 6
ctagccatccttcttccctattattctttcctttcccaaattagctctttaatcatggaaagggaccaggcaaagcctctagcaccggtttcggaccttccgagtagcgacgatggcgaggtagctttgcatcttaagcaagttcgacgcaaaaaatttgtcaagtgtttcggttccgttgcagctctagcgatcattctagctgtggtgatcataattttgattttcacagtgtttcgagtcaaggacccgatcatcaacatgaacggtgtcacggttacgagcctggagctgatcaatggcacaatcccgaagccgggttccaacatctccgtgacggctgacgtttcggttaaaaatcccaacgtagcatcatttaattataggaataccactacaatgctttactactatggcaaggtagtaggagatgcccgaggccctccgggccgtgcaaaggcccatcggatcgtgcgaatgaatattaccattgatattatcgtggatcggatcttggccagtccgaatttggtcatggatgttcggtccgggatgttgaccatggtcagctactcgagagttgggggaaggatcaatatattgaacattataaagaggcatgttactgtgaagatgaattgctccatgactatcaacattttcagccaagcaattcaacagcaaaaatgcaagcggcaggttgatgtctaagctacgtagagtgtaaatgaaacattatgagagagattatatagtttatgtagtacgattataattttggtttttttttgtatattaattacatattgctagtgaactatatgattttaaaatttaatttggggcaaattgatacacggcagaatagtatttttcatttatgtaccacatcacacattgagtgaaagtttaggagccaaatgtgttattaacccctatattaaagatgcataatatgtaaatgctataattttttccatgaactatattattggagtttcgatttaattc

SEQ ID NO: 6 - protein, 218 aa, Gossypium hirsutum
  misc_feature: Ceres CLONE ID no. 1866925
MERDQAKPLAPVSDLPSSDDGEVALHLKQVRRKKFVKCFGSVAALAIILAVVIIILIFTVFRVKDPIINMNGVTVTSLELINGTIPKPGSNISVTADVSVKNPNVASFNYRNTTTMLYYYGKVVGDARGPPGRAKAHRIVRMNITIDIIVDRILASPNLVMDVRSGMLTMVSYSRVGGRINILNIIKRHVTVKMNCSMTINIFSQAIQQQKCKRQVDV

SEQ ID NO: 7 - DNA, 591 bp, Populus balsamifera subsp. trichocarpa
  misc_feature: Encodes the peptide sequence at SEQ ID NO. 8
atgaaagctgaatctcctaagaaacacaaacgcagaaacatttgtttaggggtgaccgcagctgtgatcctctttatttttctgcttttgctcatcttggggttaacggtgttcaagcccaaacaaccgacaaccaccgtggattccacctccatcagtgacatgaaggtttcttttgacatagccaggctaagagtggatgtaaacgtgagtcttgacgtggatctctctatcaagaatccgaacaaggtgagtgtcaagtacaagaacagctctgcttttctgaattatagaggccaagttgttggtgaagctccaattcctgcgggtaagattttagcagacaaaacacaacccataaacgtaaccgttacacttatggcagatcggttgttgtctgattcgcagtttttttctgatgtcatggctggtactataccctttaataccttgaccaagatttctggaaaagctagcgtttttaatctgtttaatgtacatatcacttctaccagtagttgtgatcttcttgtttttgtttctaatagaactataggggatcaaaagtgcaagtataagacaaaattgtag

SEQ ID NO: 8 - protein, 196 aa, Populus balsamifera subsp. trichocarpa
  misc_feature: Bit score of 403.4 for HMM based on sequences of Figure 2
MKAESPKKHKRRNICLGVTAAVILFIFLLLLILGLTVFKPKQPTTTVDSTSISDMKVSFDIARLRVDVNVSLDVDLSIKNPNKVSVKYKNSSAFLNYRGQVVGEAPIPAGKILADKTQPINVTVTLMADRLLSDSQFFSDVMAGTIPFNTLTKISGKASVFNLFNVHITSTSSCDLLVFVSNRTIGDQKCKYKTKL

SEQ ID NO: 9 - DNA, 1172 bp, Glycine max
  misc_feature: Encodes the peptide sequence at SEQ ID NO. 10
gaatgtcaaagtgtcaactgtgaatgtagttagttcttctgttctgtcacaagccaaaaagcaataccaccacgccgcttttaatggcagttctgttgaacagcctgtaaagtgtaatcctacgcatccccgttcaacagtttctcttctcaattttcgtgactgccgactatcatttgataacggatcctttgaaggattgcatttttgagagatttcatctttgctttgcgggggatgtatgcgaccaacacccaccccagataacctctattgcattcccaggctgataagaaggaacaagcagaatgaaggtaggatctggtaaagggagaaaagtgtgcctgacggtgacaggtgttgtgattgcaattgtattgctaattgtgatactagcgttgacagtgttcaaagccaagcatcctgttaccacagtggactcaacgaagctagaggactttcacgtgagcttggatccagtaaaactaagggtagatttgaatgtgaccctgggagtggatgtctcagtgaagaacccgaacaaggtgggattccagtattcagacagcactgcccacctcaattacagagggcagctgataggtgaagtcccgatctctgccggagagatttcatccggtgagaccaaaggattcaatctcaccctcaccattatggccgaccgtttgctctccaattctcagcttttatctgatgtcacatctggtacattgcccctaaacactttcgtgaggatgtctgggaaagtcagcatcttaggctttatcaaagtccatgtggtttcctccacttcttgtgatgttgcaattaatctttctaatggaactgttgggaaccaagagtgccagtacaagacaaaactttgattagaggtttttagttcaagagtgaaactagactctagattagtagactatacacgtattgcttaatgatgaattaattttgttcatatttcactttgtttcatttgtagttgtgatttgtatcatactagactgtctcgacctacactctccctttaagttaagctccacatctttatgctatttgtagttttaattaatatgtaattacagggtatttctttccttccacttttaattttattaaggaattggtaccctgctataattaatagtatttttttt

SEQ ID NO: 10 - protein, 192 aa, Glycine max
  misc_feature: Ceres CLONE ID no. 472323
MKVGSGKGRKVCLTVTGVVIAIVLLIVILALTVFKAKHPVTTVDSTKLEDFHVSLDPVKLRVDLNVTLGVDVSVKNPNKVGFQYSDSTAHLNYRGQLIGEVPISAGEISSGETKGFNLTLTIMADRLLSNSQLLSDVTSGTLPLNTFVRMSGKVSILGFIKVHVVSSTSCDVAINLSNGTVGNQECQYKTKL

SEQ ID NO: 11 - protein, 192 aa, Medicago truncatula
  misc_feature: Public GI ID no. 92896037
MKAGAGKGRKACLIVTTVFIAIVLLIVILAFTVFKSKHPVTTVNSLKLRDFDVNLDIAKLRVDLNVTLDVDVSVKNPNKVGFKYSNTTAHLNYRGQLIGEVPISAGDISSGETKGFNLTLTFMADRLLSNSQLFSDITSGTLPLNTFLTIFGKVNILGFIKVHVISSASCDFAVNTSNKTVGNQECQYKTKL

SEQ ID NO: 12 - protein, 226 aa, Solanum lycopersicum
  misc_feature: Public GI ID no. 94549041
MVERDQVRPLAPASDRPHSSDDDDTTLNIKKRFHQRRCFKYCACVSTFVFLVAIIIIILIFTVFKIKDPIITMNGVTIEKLDLVNTSGTLLPIPKPGSNMTIKADVSVKNPNYSSFKYSNTTTTISYRDAVIGEARGPPGKSKARKTMRMNVTIDIMTDKIMSHPGLQDDISSGLLTMNSYTSVGGRVKLLNMIKKYVVVKMNCSITVNITSQSIQDQKCTKKVKL

SEQ ID NO: 13 - DNA, 681 bp, Sorghum bicolor
  misc_feature: Encodes the peptide sequence at SEQ ID NO. 14
atggcggccacctccacctccggcagcgtcctcccaacacacaccaccccgtccgcccccacctaccctgcttcttcctccaccaccaagcccccgccgcacccacgccgccgctgcctctgcatctgcctcctcataaccctcgccctcctaaccgcgctcgccataaccctcctcgtcctcttcctcaccgtcctcaaagtccgagacccaaccacgcgtctcatctccacccagctctccggcatcgccccacgcctaaccttcccggcggtctcactccagctcaacgtcacgctcctcctcgtcgtgtccgtgcacaacccgaacccggcgtccttcgcgtacgactccggcggccacacggacctcacctaccgcggcgcccacgtcgggtccgcggagatcgacccgggacggatcccgagccgcggcgacgggaacgtcagcctcgccctcacgctccaggccgaccgcttcgcggacgacctcccgcagctcctcggcgacgtcgaggccggcgccgtgccgctcgaggccagcaccaggatccccgggaaggtcaacatcttcggcctgttcaagcgcagcgccgtcgcgtactccgattgtagcttcgtgtttggtgtcgctgagatgcgggtgcgctcccagcagtgccgagatcgcaccaagctctag

SEQ ID NO: 14 - protein, 226 aa, Sorghum bicolor
  misc_feature: Bit score of 455.4 for HMM based on sequences of Figure 2
MAATSTSGSVLPTHTTPSAPTYPASSSTTKPPPHPRRRCLCICLLITLALLTALAITLLVLFLTVLKVRDPTTRLISTQLSGIAPRLTFPAVSLQLNVTLLLVVSVHNPNPASFAYDSGGHTDLTYRGAHVGSAEIDPGRIPSRGDGNVSLALTLQADRFADDLPQLLGDVEAGAVPLEASTRIPGKVNIFGLFKRSAVAYSDCSFVFGVAEMRVRSQQCRDRTKL

SEQ ID NO: 15 - DNA, 1002 bp, Zea mays
  misc_feature: Encodes the peptide sequence at SEQ ID NO. 16
tccaagacccgcccgcatcatcgtcctcctccgcgctcccattccagttccagcctccaccccatggcgacggcgacggcgaccgcagtaccgtacaacgccgcggcggcggcagcagcggccgggaaggaggagacgacgaggccgatcgccgtcgcgtccccgaccgtccacccggcggcgacgggggacgaggaggaggccgcgaccgccaccaggcgctggcggccgacgcagtacctccgcaagcggcggtgcttgctctggtgctgcggctgctgcgccacctcagtggtcctcctgggcatcaccgtgctggtgctggcgctgaccgtgttcagggtgaaggaccccgtgctgaccatgaacggggtgacgctggagggcgtggacggcggccccggctcgacgccggagcacccggtgtccgtgaacgcgacgctcagcgccgacatctccatcaagaaccccaacgtggcgtccttcgcgttcggccgcagcgagacggacttctactacggcggggagacggtgggcgtggcgtacgcgccccagggcgaggtgggcgccgaccgcaccgtgcgcatgaacgtgacgctggacgtgctcgccgaccgcgtctcgcccagcgtcaacgccacggacctcatcttcggccaggagtacaaccttaccagctacacggagatcgccgggagaatcagcgtgctcggcatctacaagagggacctggacatcaagatgaactgctccatcacgctcgaggtgggggccttcaccaccgtgcagagcaaagccaccgactgcgttgcaaatgtcagctgattttttttcggttagctggtcgttagtcaccctccttccttcattctgtatgtatgcaagatatagagtgttgtatgttgcttttctttttttttgtcagagattagtgactgttgtaactcatatacgtctataaatcctgatgatgtgaggcttatgtaagttcaggccacaatt

SEQ ID NO: 16 - protein, 253 aa, Zea mays
  misc_feature: Ceres CLONE ID no. 1469145
MATATATAVPYNAAAAAAAAGKEETTRPIAVASPTVHPAATGDEEEAATATRRWRPTQYLRKRRCLLWCCGCCATSVVLLGITVLVLALTVFRVKDPVLTMNGVTLEGVDGGPGSTPEHPVSVNATLSADISIKNPNVASFAFGRSETDFYYGGETVGVAYAPQGEVGADRTVRMNVTLDVLADRVSPSVNATDLIFGQEYNLTSYTEIAGRISVLGIYKRDLDIKMNCSITLEVGAFTTVQSKATDCVANVS

SEQ ID NO: 17 - DNA, 719 bp, Musa acuminata
  misc_feature: Encodes the peptide sequence at SEQ ID NO. 18
accgccggtaatggactccaaggaacatcgccaccatcggtggtgctctcgacgctgcgtaatcatctccgttgcagtaatccttggcatcgccctcctcctcctcatccttggcctcaccgtctttcggccccgccacacagtcaccaccatcaattcggtgcatctcggcgccctccgtgtcgggctcgacgtcccccatctttccgtcgatcttaacgtcaccctagacctcgatataacggccaccaaccctaaccgcgcgagcttccgctacgacaccggaaacgccgagctcttctaccatgggggcctcgtcggcgaggcggttatcccgccggggagggtcggagccgagggttcggtgcggaccaacgtatctctcacggtgatggccgaccgactcatctccgacgcgaccctgtacaaggacgtgatctcggggtcggttcccttctccaccaatacgagactccccgggacggtgacgattcttggggtgttcaagcaccacatggtggcgtacaccatgtgcaacatcaccgtgagcgttcagagccgatcggtggagaactccgattgcaggtataagacaaaattctgagcctttttgttcctgattggagtcacggacgagcttccttcgagtaaaattgtgtagaatacatgtgcttcatctctggatttgctcatgaaatgaattgattattgtgactgcc

SEQ ID NO: 18 - protein, 197 aa, Musa acuminata
  misc_feature: Ceres CLONE ID no. 1731181
MDSKEHRHHRWCSRRCVIISVAVILGIALLLLILGLTVFRPRHTVTTINSVHLGALRVGLDVPHLSVDLNVTLDLDITATNPNRASFRYDTGNAELFYHGGLVGEAVIPPGRVGAEGSVRTNVSLTVMADRLISDATLYKDVISGSVPFSTNTRLPGTVTILGVFKHHMVAYTMCNITVSVQSRSVENSDCRYKTKF

SEQ ID NO: 19 - protein, 220 aa, Vitis vinifera
  misc_feature: Public GI ID no. 147832282
MVEREQVRPLAPASHRLSSEDDKVTNHLSRLRRRRCIKCWGCIAATILIQAAVVIILVFTVFRVKDPVIKLNGFTVDKLELINGTTTPGPGVNMSLTADVSVKNPNFASFRYKNTTTTLFYSGTVIGEARGPPGQAKARRTMKMNVTIEIILDSLMSNPSLLTDISSGILPMNTYSRVPGRVKMLKIIKKHVVVKMNCSVTVNITSRSIQEQKCKRDVNL

SEQ ID NO: 20 - DNA, 802 bp, Panicum virgatum
  misc_feature: Encodes the peptide sequence at SEQ ID NO. 21
atccccacaatcccaggcgcccgaatccaccaatcaatctatccatctcaatggcctcctcctctgccgccggtaacggcaccggcggcatcctccccacgcacaccgccgcccccgcctggccttcaacttccaagcccccgcccaccaaggaccctcgccggcggcggcgctgcctctgcgtgtgcctcctcgtcaccctggccgtcctgctcgcgctcgccatcaccctgctcgtcctattcctcaccgtcctcaaggtccgggaccccaccacgcgcctcgtctccacgcgcctggcgggcgtcgccccgcgcctcaccttcccggccgtctccctccagctcaacgtcacgctgctgctcaccgtggccgtgcacaacccgaacccggcctccttcgcctacgacgccgggggccacaccgacctcacctaccgcggctcgcacgtcggcgacgcggccatcgacccgggccgcatccccagcaagggcgacggcgaggtcaagttggcgctcacggtgcaggcggaccggctcgcggccgacctggcgcagctggtggcggacgtggagagcgggtcggtggccatggaggccagcaccaggatccccggcagggtcaccatcctgggcatcttcaagcgccacgccgtcgcctactccgactgcagcttcgtcttcggcgtcgcggagatggcggtgcgcagccagcagtgccatgatcgcacaaagctctgatgattgatgaatctgaattgatgatccatcatccatccaaggaagcatctggttgccgtgcc

SEQ ID NO: 21 - protein, 229 aa, Panicum virgatum
  misc_feature: Ceres CLONE ID no. 1874239
MASSSAAGNGTGGILPTHTAAPAWPSTSKPPPTKDPRRRRRCLCVCLLVTLAVLLALAITLLVLFLTVLKVRDPTTRLVSTRLAGVAPRLTFPAVSLQLNVTLLLTVAVHNPNPASFAYDAGGHTDLTYRGSHVGDAAIDPGRIPSKGDGEVKLALTVQADRLAADLAQLVADVESGSVAMEASTRIPGRVTILGIFKRHAVAYSDCSFVFGVAEMAVRSQQCHDRTKL

SEQ ID NO: 22 - protein, 235 aa, Oryza sativa subsp. japonica
  misc_feature: Public GI ID no. 115483889
MASTTAAAAGNGSGSILPTHTIAATAPPFRTHKDADLESRRRRRRRRCLCCCLLVTLVVLLVLAITLLVLFLTVLRVRDPTTRLVSTRLIGLSPRLSFPAMSVQLNVTLLITVAVHNPNPASFTYATGGHTDLTYRGAHVGDAEIDPGRIPSRGDANVTMALTLQADRFAGDLTQLVTDVMGGSVALEASTRIPGRVAILGVFKRHAVAYSDCHFVFGVTEMAVRSQQCSDRTKL

SEQ ID NO: 23 - DNA, 669 bp, Miscanthus x giganteus
  misc_feature: Encodes the peptide sequence at SEQ ID NO. 24
gtccggcaaggggcacaacgaactacgcctgcctcttcccggttcccctttctcccctcccctatttcatttgattcatccactcgccttcgccgcaccccccgtccctctcccccaaatcccaatccatctaatggcggccacctccggcagcagcgtcctcccgacgcacacagccccgtccgcccccgcctaccctgcctccaccaagccctccccgaacccacgccggcgctgcctctgcatctgcctcctcataaccctcgccttcctcgccgcgctcgccatcacgctgctcgtcctcttcctgacggtcctgcgcgtccgcgaccccacgacccgcgtcgtctccacgcagctctccggcatcgccccgcgcctcaccttcccggccgtctcgctccagctcaacatcacgctcctcctcgtggtgtccgtgcacaacccgaacccggcctcgttcgcgtacgcctcggggggccacacggacctcacgtaccggggcgtccaggtcggggccgcggagatcgacccgggccggatcccgagccgcggcgacggcaacgtcagcctcgcgctcacgctccaggccgaccgcttcgcgtccgacctgccacagctgctcagcgacgtcgaggccggcgccgtgccgctggaggccgccaccag

SEQ ID NO: 24 - protein, 178 aa, Unknown
  misc_feature: Miscanthus x gigantus
MAATSGSSVLPTHTAPSAPAYPASTKPSPNPRRRCLCICLLITLAFLAALAITLLVLFLTVLRVRDPTTRVVSTQLSGIAPRLTFPAVSLQLNITLLLVVSVHNPNPASFAYASGGHTDLTYRGVQVGAAEIDPGRIPSRGDGNVSLALTLQADRFASDLPQLLSDVEAGAVPLEAAT

SEQ ID NO: 25 - protein, 231 aa, Arabidopsis thaliana
  misc_feature: Public GI ID no. 18379145
MPPPPSSSRAGLNGDPIAAQNQQPYYRSYSSSSSASLKGCCCCLFLLFAFLALLVLAVVLIVILAVKPKKPQFDLQQVAVVYMGISNPSAVLDPTTASLSLTIRMLFTAVNPNKVGIRYGESSFTVMYKGMPLGRATVPGFYQDAHSTKNVEATISVDRVNLMQAHAADLVRDASLNDRVELTVRGDVGAKIRVMNFDSPGVQVSVNCGIGISPRKQALIYKQCGFDGLSV

SEQ ID NO: 26 - protein, 235 aa, Arabidopsis thaliana
  misc_feature: Public GI ID no. 15232445
MSDFSIKPDDKKEEEKPATAMLPPPKPNASSMETQSANTGTAKKLRRKRNCKICICFTILLILLIAIVIVILAFTLFKPKRPTTTIDSVTVDRLQASVNPLLLKVLLNLTLNVDLSLKNPNRIGFSYDSSSALLNYRGQVIGEAPLPANRIAARKTVPLNITLTLMADRLLSETQLLSDVMAGVIPLNTFVKVTGKVTVLKIFKIKVQSSSSCDLSISVSDRNVTSQHCKYSTKL

SEQ ID NO: 27 - protein, 235 aa, Oryza sativa subsp. indica
  misc_feature: Public GI ID no. 125533193
MASTTAAAAGNGSGSILPTHTIAATAPPFRTHKDADLESRRRRRRRRCLCCCLLVTLVVLLVLAITLLVLFLTVLRVRDPTTRLVSTRLIGLSPRLSFPAMSVQLNVTLLITVAVHNPNPASFTYATGGHTDLTYRGAHVGDAEIDPGRIPSRGDANVTMALTLQADRFAGDLTQLVSDVMGGSVALEASTRIPGRVAILGVFKRHAVAYSDCHFVFGVTEMAVRSQQCSDRTKL

SEQ ID NO: 28 - protein, 233 aa, Oryza sativa subsp. japonica
  misc_feature: Public GI ID no. 115487056
MASTTATAAGNGSGSILPTHTTTAPPFRAHKDADLESTTRRRRRRCLCCCLLVTLVVLLVLAITLLVLFLTVLRVRDPTTHLVSTRLTGLSPRLSFPATSVQLNVTLLITVAVHNPNPASFTYATGGHTDLTYRGAHVGDAEIDPGRIPSRGDANVTMALTLQADRFAGDLTQLVSDVMGGSVALDASTRIPGRVAILGVFKRHAVAYSDCHFVFGVTEMAVRSQQCSDRTKL

SEQ ID NO: 29 - DNA, 670 bp, Arabidopsis thaliana
  misc_feature: Expected sequence
attgttgttttgtatataaaatccctaaattcccgttttatgtaacggacataataagccgcgtacggcgtatccggcaaatcggcccagctcgagccatcatcaatctacggaggagatccatctctctctctctctccatatcgcaacaacactgctgaagatttcgggggttgtcgacggagctggaatctagctttttcttctccagatctctaaaccgattctcaccggttaattggtattataaagccatggttcttgatgggcttgtatcttcaccgtcaaggagacaacaatgtctaaagaagcagtgggacgagttgggtagctggtcgactcttattcagaggcatcaatatctcttaacagctttggctcttttggctttcctctgtactgtttatctttactttgcagtcactttaggcgctaggcactcgtcctgctatggcttgaccgggaaagacaaggcaatgtgtcaattacaacttgttcaagctctctccaaagggaaacttaaattcttctagactgtttatctatcggtgaaagtttgaaacccccacatttattgttcttgtactaaaacattctttttcttctttattctataatgcaatttgtgtgtattattacatggtgaaataaaaccagagattcataattg

SEQ ID NO: 30 - DNA, 670 bp, Arabidopsis thaliana; identical to SEQ ID NO: 29
  misc_feature: Inplanta sequence

SEQ ID NO: 31 - protein, 92 aa, Arabidopsis thaliana
  misc_feature: Ceres CLONE ID no. 32430
MVLDGLVSSPSRRQQCLKKQWDELGSWSTLIQRHQYLLTALALLAFLCTVYLYFAVTLGARHSSCYGLTGKDKAMCQLQLVQALSKGKLKFF

SEQ ID NO: 32 - protein, 103 aa, Picea sitchensis
  misc_feature: Public GI ID no. 116788849
MVHESITSPHRRAQNPVSPLSASLRRQFQPRDELDSWMALFRRHQFLLIMLAILTFLCTVYLYFAITLGAAQSCSGLSGTQKALCHLEQMKTSMPRKGKLKFF

SEQ ID NO: 33 - DNA, 526 bp, Gossypium hirsutum
  misc_feature: Encodes the peptide sequence at SEQ ID NO. 34
atggtaacgtcccccttccactgagaaataaaggagaagacaaatggagaagaggagttaaaaagatcgattaaatctatatactcattgtttgcttcttccctttgatcttcctaccttgattttctcatctaccacattggttgaggacttttaatcgtctttctaagaaatggttctggacggaattgtatcatctccccttcgaaggtcagcatcaacgaggaggcaatcctcgagagatgaattcggtagctggtcaacacttgttgagaggcataggttcctcttaacagcattgggattgttggcattcctctgtactatctacctttattttgctgttactttaggagccacggatacatgttctgggttgaaaggaacagagaaagcaacatgtaatctgcagcatgtaagctccactctctcccatgggaaactcaaattcctgtgacattcttcatgctatgtatgttacaagttgcttttattaattaaacttataatctgagcatcttaatat

SEQ ID NO: 34 - protein, 94 aa, Gossypium hirsutum
  misc_feature: Ceres CLONE ID no. 1808741
MVLDGIVSSPLRRSASTRRQSSRDEFGSWSTLVERHRFLLTALGLLAFLCTIYLYFAVTLGATDTCSGLKGTEKATCNLQHVSSTLSHGKLKFL

SEQ ID NO: 35 - DNA, 285 bp, Populus balsamifera subsp. trichocarpa
  misc_feature: Encodes the peptide sequence at SEQ ID NO. 36
atggttcttgatagcatgataacttctcctcaccggagatcaccatctttccggaagccattcccacgggatgagttgggtagctggtcaacactccttcggcgacaccgtttcctcttaacagccttcgctctcttggctttcttatgcacaatctatctctacttcgctgttaccctaggggccactgaatcatgttctggactgacaggtaccaaaaagacattatgtcgtttggagctggcaaaggattctgtaggcaatggaaaactcaaatttttctag

SEQ ID NO: 36 - protein, 94 aa, Populus balsamifera subsp. trichocarpa
  misc_feature: Bit score of 185.4 for HMM based on sequences of Figure 1
MVLDSMITSPHRRSPSFRKPFPRDELGSWSTLLRRHRFLLTAFALLAFLCTIYLYFAVTLGATESCSGLTGTKKTLCRLELAKDSVGNGKLKFF

SEQ ID NO: 37 - DNA, 599 bp, Glycine max
  misc_feature: Encodes the peptide sequence at SEQ ID NO. 38
aaaccgcgtacggcaccatccggctaatcgccaccgctcgaagctacggaggagattgattcaacctctctctatctatatcggaaacgtctaacggcggactaatttacgtgtcgacggagctagaagcagcttatcttctccagatctctaaccgattctcaccgcttcctgtttcaccggttgggtgatcaaagcaatggttcttgatgggattgtatcttcaccattgaggagacaccagtctctaaagaaacagtgggaagatttgggtagctgctcaaccgttgttaaccggcatcgatatttattaacagcattgcttctcttgggctttctctgcacggtttatctttactttgctgtcactttagatgctaggcacaactcctcctgctacggcttggctgggaaagagaaggcaatgtgtcaagccatctctaaagggaaactcaaattgttttgataaattgttcttgtgccactacattcttttttgttttattgattctataatgccatttgtgtgtaattgtttcattacatggtgaaaaaccagagatttgtaacctaaacatcaaaaaacattttcatttctg

SEQ ID NO: 38 - protein, 88 aa, Glycine max
  misc_feature: Ceres CLONE ID no. 1246448
MVLDGIVSSPLRRHQSLKKQWEDLGSCSTVVNRHRYLLTALLLLGFLCTVYLYFAVTLDARHNSSCYGLAGKEKAMCQAISKGKLKLF

SEQ ID NO: 39 - DNA, 375 bp, Solanum lycopersicum
  misc_feature: Encodes the peptide sequence at SEQ ID NO. 40
atggctcttgacagcataataacttctcctcataggaggtcacagacacaaacagcattttcatctactgatccaaagaagaatcaatactctcgaagcaatgagcttgggagctgttcaacagtacttcagcgccaccggttccttctaattgcacttggcctcctagcttttctctgcactatctatctttactttgcagtcactctcggggctggtgactcctgttctgacttgacagggactcaaaaagcagcatgctatgtggagcacgggaaagcacacatggacaaaggaaaactcaagggaggttgggactatgaagttgactcttatgctgtttgctataaaagtcttttaggactttctgcatag

SEQ ID NO: 40 - protein, 124 aa, Solanum lycopersicum
  misc_feature: Bit score of 187.2 for HMM based on sequences of Figure 1
MALDSIITSPHRRSQTQTAFSSTDPKKNQYSRSNELGSCSTVLQRHRFLLIALGLLAFLCTIYLYFAVTLGAGDSCSDLTGTQKAACYVEHGKAHMDKGKLKGGWDYEVDSYAVCYKSLLGLSA

SEQ ID NO: 41 - DNA, 758 bp, Zea mays
  misc_feature: Encodes the peptide sequence at SEQ ID NO. 42
ccctcattgcccgtcgccaatattcggacggttcaccggggccacgcctctcttctctccctttccaagggccgccgcatccactccgccccccgcctcgcagtgcctccccaccgccctccgcgcccggcaggcgacgtcccgttccagatccaggattcaaacaaaccagtacattaccaccatggttcttgattcattatcatcacctcacaggaggtcccaaaacacattcttcgtgtcatctgcaaaaaagcctcagtcatctcgtgacgacagttggtctgcactggttgagcgacaccggtttctcctgacgacactccttgttcttgccttcctgtgcactgtctatctgtactttgcagtaaccttgggggcatcagatgcttgcactggattgacaggagcagagaggattgagtgccaggcaagatcagtgctgcaacatggaaagttgaaattccgatgactttggagactcattgatgaagaccaaattattgagttttatatgcactcgggtcacatgacaaattcttgataccatgtatctgtatatggtggggtggctgatgttattggaccacaaaatttgctgtactggtagaggcaaatctaatagttagggttgagcttgtggaaacgggtatactggggtaggcccatattgttcttttaccttggtgtgcgaaaaacatcatggcgtctgtaattcttccaggagatattggtattgtctcccttgagacatactc

SEQ ID NO: 42 - protein, 95 aa, Zea mays
  misc_feature: Ceres CLONE ID no. 1678697
MVLDSLSSPHRRSQNTFFVSSAKKPQSSRDDSWSALVERHRFLLTTLLVLAFLCTVYLYFAVTLGASDACTGLTGAERIECQARSVLQHGKLKFR

SEQ ID NO: 43 - DNA, 685 bp, Musa acuminata
  misc_feature: Encodes the peptide sequence at SEQ ID NO. 44
gtaacccctcgttggatttctgatcgtttcttcgttggtcgcaccggcacaaaattttcaatcacactcagtattcttaagattgcaatatcaatgaatttcttaccactaaaggtttttgaagcaaagtttgattcaagatgggtctcacacatggaactttgaagcaaccgtgtaggactttaggcagcttagaccatcttgacagatggttcttgattcactatcatctcctcataggagatcacagaacacagttttcctggcatccccgtccaagaaacagcagtcgggcttcaatgagcctggtagttggtccactatttatgagcggcacaggtttctcttgacgatgctagctctattggcattcctatgcaccatctatttgtactttgcagttactttgggagccacaggctcatgctcaggaatgtcaggagctgagaaagctctttgccaggccaaatcttcattgcacaagggaaaattgaaattcttttgaccagcaagtgatctcgagcatctagaaaagcaatgcctctgggattaatgatcaattcataacaattcataaacactatatgatatgggggcattgcatgtttggtttcctgcagcaaattataagatttgtgggttgtggtttaccgatgataaatgtgtatttgatttgatgtgttag

SEQ ID NO: 44 - protein, 98 aa, Musa acuminata
  misc_feature: Ceres CLONE ID no. 1727075
MVLDSLSSPHRRSQNTVFLASPSKKQQSGFNEPGSWSTIYERHRFLLTMLALLAFLCTIYLYFAVTLGATGSCSGMSGAEKALCQAKSSLHKGKLKFF

SEQ ID NO: 45 - DNA, 851 bp, Panicum virgatum
  misc_feature: Encodes the peptide sequence at SEQ ID NO. 46
ctcgaccgtcgccaatattcggacggttcaccgggacgaggtaacccctctggccctctccaattccaagggccgccgcgtcctctccgctccgccccacctcgtcgtgcctgcccaccgccctcccctccccgccggccggcctcgccccgatccagatccaggattcaaactaataagtacactgccaccatggttctggattcattatcatcccctcaccggaggtcccagaacacattctttgtgtcatccacaaagaagcctcagccatctcgggatgacagttggtctgcactgcttgagcggcaccggttcctcttgacaacgcttgttgtgcttgccttcctgtgcaccatctatctgtacttcgcagtaaccttgggggcatcaaatgcttgcgctggattggcaggggcggagaggattgagtgccaggcaaaatccgttctgcaacatggaaagttgaaattcctctgacgctgagttgttttgagactcattggtgaagaccaaaatattgagttttatatgcgctctggtcacaggtcttattcttcataacttgaatatgtatattttgggtggtgggtggtgtgtattcatgctcatgtacttggactgcgagatttactgtactgtacccccgtagaggcaacaaatctaatagtgatagtgctgagttatggaaacgttatgatgaagtaggtcaccatattattcttttaccatgatgtactgaaaacatcgtggcgccattattcatcaaggagatcttgatattgtctcccttacatgtaactcgaaattaatttgaaactgtgctgcctgagtatatacttattgatcag

SEQ ID NO: 46 - protein, 95 aa, Panicum virgatum
  misc_feature: Ceres CLONE ID no. 1871675
MVLDSLSSPHRRSQNTFFVSSTKKPQPSRDDSWSALLERHRFLLTTLVVLAFLCTIYLYFAVTLGASNACAGLAGAERIECQAKSVLQHGKLKFL

SEQ ID NO: 47 - protein, 97 aa, Oryza sativa subsp. japonica
  misc_feature: Public GI ID no. 115475611
MVLDSLSSPHRRSQNTFFLSSPKKLQSSKDDVGSWSALVERHRFLLTTLVVLVFLCTIYLYFAVTLGAPDACSGLAGTEKAVCRAKSALRHGKLKFF

SEQ ID NO: 48 - DNA, 285 bp, Populus balsamifera subsp. trichocarpa
  misc_feature: Encodes the peptide sequence at SEQ ID NO. 49
atggttcttgatagcatgataacttctcctcacctgagatcaccatccttccggaaacaattcccacgggatgatttgggtagctggtcaactctccttcagagacaccgtttcctcttaacagccttggttctcttgggtttcttatgcacaatctatctctacttcgctgttactctaggggccactgaatcatgttctggatttaccggaaccaaaaaggcattatgtcatgtggagctggcaaaggattctgtaggccacggaaaactcaagtttttctag

SEQ ID NO: 49 - protein, 94 aa, Populus balsamifera subsp. trichocarpa
  misc_feature: Bit score of 181.3 for HMM based on sequences of Figure 1
MVLDSMITSPHLRSPSFRKQFPRDDLGSWSTLLQRHRFLLTALVLLGFLCTIYLYFAVTLGATESCSGFTGTKKALCHVELAKDSVGHGKLKFF

SEQ ID NO: 50 - DNA, 770 bp, Zea mays
  misc_feature: Encodes the peptide sequence at SEQ ID NO. 51
cggatttccccctcctcgaccgtcgtcaatatcgcacggttcaccggccacgctctctcgctttccaaggaccgccgggtccgctccgtcccccgtctggcagtggcctcctcgccgccctcggcgccgcctggccacgccttatccagatccaggattcaaagaaaccattacttcaccaccatggttcttgattcattgtcatctccgcacaggaggtcccaaaacatattcttcgtgtcatcatcaaaaaagcctcagtcatctcgtgatgacagttggtctgcactggttgagcggcaccggttcctcctgacaacacttcttgtgcttgccttcctatgcagtatctatctgtactttgcagtgaccttgggggcatcagatgcatgcagtggtttgacaggaggggcacagaagatcgagtgccaggcaagatcagtgctgcaacatggaaagttgaaattccgctgacttcggagtctcattgatgaagaccaaattattgagtttatttgcactctggtcacaggtcttattcttcataccatgaatctgtatattattggtggtggctggtttgtattcatgctgatgttattgggccacaacatttattgtactggtagagataaatctaatagtgatagcgttgagtttgtggaaagtggaaactggtataagggtaggtctatattgttcttttaacaatcatggtgcctataattcgtccaggagaccttggtattgtctcccgtgtgacatgtgac

SEQ ID NO: 51 - protein, 96 aa, Zea mays
  misc_feature: Ceres CLONE ID no. 392748
MVLDSLSSPHRRSQNIFFVSSSKKPQSSRDDSWSALVERHRFLLTTLLVLAFLCSIYLYFAVTLGASDACSGLTGGAQKIECQARSVLQHGKLKFR

SEQ ID NO: 52 - DNA, 459 bp, Sorghum bicolor
  misc_feature: Encodes the peptide sequence at SEQ ID NO. 53
atggttcttgattcattatcatctcctcacaggaggtcccaaaacacattcttcgtatcatctgcaaaaaagcctcagtcatctcgtgatgacagttggtctgcactggttgagcggcaccggttcctcctgacaacacttcttgtgcttgccttcctgtgcactatctatctgtactttgcagtaaccttgggggcatcagatgcttgcattggattggcaggggcagagaggattgagtgccaggcaagatcagtgctgcaacatggaaagtcttattcttcataccatgaatctgtatatttttggtggtgggtggtttgtattcacgctgatgttattggaccacaaatttactgtactggtagaggcaaatctaatagtgacagtgttgagtttgtggaaactggagatcttggtattgtctcccgtgtgacatgtgactcacagttaatttga

SEQ ID NO: 53 - protein, 152 aa, Sorghum bicolor
  misc_feature: Bit score of 168.1 for HMM based on sequences of Figure 1
MVLDSLSSPHRRSQNTFFVSSAKKPQSSRDDSWSALVERHRFLLTTLLVLAFLCTIYLYFAVTLGASDACIGLAGAERIECQARSVLQHGKSYSSYHESVYFWWWVVCIHADVIGPQIYCTGRGKSNSDSVEFVETGDLGIVSRVTCDSQLI

SEQ ID NO: 54 - DNA, 603 bp, Arabidopsis thaliana
  misc_feature: Encodes the peptide sequence at SEQ ID NO. 55
atttggtcgacggagctggaatcagctattctttcttcttctccctaatcgtttcttttagcagctctttctctctcacccgaaggttggtgttaagctggaaaggttggtcttttagaagatatggttcttgatgggattgtatcatcaccattacggaggccacatgctcttaagaagcaatgggatgacttgggtagctgctctactgttgtccagaggcatcgtttccttttgactgctatgcttcttttggcattcctctgcaccatttatatctactttgccgtcacactaggcgctaggcacttgttgtgctcagggatgactggaaaagacaaggcaatgtgtcaaatggaacacatccaagccagtttctccaacggaaaactgaaattcttctagagtgtcagcctaagctgattgcattatataaacatttgtctctctgttgaaatgtgtgtgtaatcgcctaaagaatggcttgtaaacccatcagaatctacacgtaccttattctactttgattatgtcccaagattcacagttattgtacaaatatgaatcttctgtaatcgaatatatatgctaaaagttttggag

SEQ ID NO: 55 - protein, 93 aa, Arabidopsis thaliana
  misc_feature: Bit score of 159.6 for HMM based on sequences of Figure 1
MVLDGIVSSPLRRPHALKKQWDDLGSCSTVVQRHRFLLTAMLLLAFLCTIYIYFAVTLGARHLLCSGMTGKDKAMCQMEHIQASFSNGKLKFF

SEQ ID NO: 56 - DNA, 593 bp, Glycine max
  misc_feature: Encodes the peptide sequence at SEQ ID NO. 57
atattgtgcggcaactgtctcatcacatctcgcgaatccaagccgaaggggaagggggtttgataaaggttgacattgaaatttgaagccatggttcttgactccattctgacatctcctcgtctcaagtctccatctttcaggagacagtttaaaaaggatgaactgggtagttggtccacacttttccagaggcatcgcttcctcttatctgctcttgttctgctaactctcctctgcactgtttatctttactttgctgtcactttaggggctagtggcacctgttctggtttgactggagcgcagaaagcttcatgccatatggagcttgttaaggattctgtggccaagggtaaactgaaaattctttgatatttcaaaatataattacagggaatattctttctttatttttgcctgagtataggtgacgtgatgaaatccaaatatagtctccggctactgtatcaatcagtgtgactgtgatatttatacatactattatttatcttgtatttttgagccaaaatgtagaatatatgtagaaagcaatttcttagatacattatgctaattttagtgattgcttc

SEQ ID NO: 57 - protein, 94 aa, Glycine max
  misc_feature: Ceres CLONE ID no. 1661096
MVLDSILTSPRLKSPSFRRQFKKDELGSWSTLFQRHRFLLSALVLLTLLCTVYLYFAVTLGASGTCSGLTGAQKASCHMELVKDSVAKGKLKIL

SEQ ID NO: 58 - DNA, 668 bp, Brassica napus
  misc_feature: Encodes the peptide sequence at SEQ ID NO. 59
acggtgacgaagacaaagcttctgctatttgggggagctaaactaggagtttttttttcattgttgacggagggagctgtaatcagctcttcttcatctccagatctcgaatcggttggtgtttaagctagaaggatcagttttttttttattgaagaaatggttcttgatgggattgtgtcatcaccactacgtaggccgcatgcgctaaagaagcaatgggaggatttgggtagcttctccactgttttcaggaggcatcgcttccttttaacagctatgctccttttggccttcctctgcaccatctacatctactttgccgtcacgttaggcgctaggcacttgtcgtgttctgatatgaccgggaaagagaaggcgatctgtcaaatggaacacgtccatgccagtttctctaaagggaggaaactgaaattcttttgaaragtgtcagccaaagctgatgcgcattataatgcaatgtgtgtgtgtaacaacaatagcytaaagaaatggsttatgtttaaccataaatgtaaaactttttgtattgtattatttgattgtgtcccaagatgattccacaaaaacattgtataaatgaatctttgtaatggaatgtaatactgaaaagattatcacacaattaaaacaaacattttggtact

SEQ ID NO: 59 - protein, 94 aa, Brassica napus
  misc_feature: Ceres CLONE ID no. 951894
MVLDGIVSSPLRRPHALKKQWEDLGSFSTVFRRHRFLLTAMLLLAFLCTIYIYFAVTLGARHLSCSDMTGKEKAICQMEHVHASFSKGRKLKFF

SEQ ID NO: 60 - DNA, 641 bp, Brassica napus
  misc_feature: Encodes the peptide sequence at SEQ ID NO. 61
atttgggaagctaaaggaggagttttttttctctttccattgtcgagggagggagctgtaatcagctcttcttcatctccagatctcgaatcggttggtgcttaagctagaaggatcagttttttattgaagcgatggttgttgatgggattgtgtcatcaccactacgtaggccacacgtgctaaagaagcaatgggaggacttgggtagcttctctactgttttccggaggcatcgcttccttttaaccgctatgatccttttggccttcctctgcaccatctacatctactttgccgtcactttaggcgctaggcacttgtcgtgttcggatatgaccgggaaagagaaggcaatctgtcaaatgggacacgtccacgccagtttctctaaagggaggaaactgaaattcttttgaagagtgtcagccaaagctgataagcattattataatgcaaatgtgtgtgtaacaacaatagcctgaagaaatggcttgtgtgttgatcataatgtatactttttgtattgtattatttgattgtgtcggaagatgattccacaaaaacattgtataaatgaatctttgtaatggaatgtaatactgaaaaagattatcacacaattaaaacaacattttggt

SEQ ID NO: 61 - protein, 94 aa, Brassica napus
  misc_feature: Ceres CLONE ID no. 1116694
MVVDGIVSSPLRRPHVLKKQWEDLGSFSTVFRRHRFLLTAMILLAFLCTIYIYFAVTLGARHLSCSDMTGKEKAICQMGHVHASFSKGRKLKFF

SEQ ID NO: 62 - DNA, 459 bp, Glycine max
  misc_feature: Encodes the peptide sequence at SEQ ID NO. 63
aaaccgcgtacggcaccatccggctaatcgccaccgctcgaagctacggaggagattgattcaacctctctctatctatatcggaaacgtctaacggcggactaatttacgtgtcgacggagctagaagcagcttatcttctccagatctctaaccgattctcaccgcttcctgtttcaccggttgggtgatcaaagcaatggttcttgatgggattgtatcttcaccattgaggagacaccagtctctaaagaaacagtgggaagatttgggtagctgctcaaccgttgttaaccggcatcgatatttattaacagcattgcttctcttgggctttctctgcacggtttatctttactttgctgtcactttagatgctaggcacaactcctcctgctacggcttggctgggaaagagaaggcaatgtgtcaagccatctctaaagggaaactcaaatt

SEQ ID NO: 63 - protein, 86 aa, Glycine max
  misc_feature: Ceres CLONE ID no. 1246352
MVLDGIVSSPLRRHQSLKKQWEDLGSCSTVVNRHRYLLTALLLLGFLCTVYLYFAVTLDARHNSSCYGLAGKEKAMCQAISKGKLK

SEQ ID NO: 64 - DNA, 668 bp, Glycine max
  misc_feature: Encodes the peptide sequence at SEQ ID NO. 65
aattacaatacaaccttcagctcttgttcacgaaacacgcgctttttcacagccttcgaagtcaaagattctggctgagcagtcctaagtaagtcattgtagaagtcatggttctagagtccattttgtcgtcttcgaagtcaccatctttcaggaggcagtttgcaaagcatgaactgggcagttggtcgacactcttgaagaggcaccgcttcctcttatttgctcttgctctactaactgtcctctgtaccatttatctatattttgcagttacatttgcagccaatgactcctgctctgggttgaatggacctctgaaagattcctgtcatatggagcatgttaaggcttctgtagccaagagtaaactgaaaggtctgagacatttctgattccaattcaagttgtgctggagagtttttatcttttcctttaggagacatgaaatcgaaatatgatttttcggctcagtgttactataatatgtattatttatctatactgtttatcttagtatttgtgagccaaaatgtacaatccatgttagaaagcaatttcttgattgctcattttggggggctgctttatgatggctaatataagttcggtgagcatcctagcagcccaaatcaaatttgagtttttacaatgttgatggttaattc

SEQ ID NO: 65 - protein, 95 aa, Glycine max
  misc_feature: Ceres CLONE ID no. 724313
MVLESILSSSKSPSFRRQFAKHELGSWSTLLKRHRFLLFALALLTVLCTIYLYFAVTFAANDSCSGLNGPLKDSCHMEHVKASVAKSKLKGLRHF

SEQ ID NO: 66 - DNA, 673 bp, Glycine max
  misc_feature: Encodes the peptide sequence at SEQ ID NO. 67
ggttacggatggttcctcttcttcctctcacatggcatgacaccagtaataataacacattccactttcccatctctggtttttacattacaatacaaccttaacgctcttgttcacgaaacacgcgctttttcacaaccttcgaagtctttttcgcaaccttcgaagtcaaaagattcaggcattctagtcccgagtaagtcattgtagaagccatggttctcgactccattttgtcgtcttctccttgtctgaagtcaccatctttcagtaggcagtttgcaaggcatgaactgggaagttggtcaacactcgtcaagagacactgcttcctcttatctgctcttgctctactaactgtcctctgtaccatttatctatattttgcagttacattcgcagccaatgactcctgctctgggttgagtggatctctgagagattcctgtcatatggagcatgttatggattctgaagccmagagtwaactgaaaggtytgagacatttatgatttccaattcaagttgtgccggagagtttttttatcttttcctttagtataatatgtactatttatccatactgtttatyttagtatttgtgagccaaaatgtacaatgtatgtagaaagcaatttbttkaatgstcatttgggggctgctttytggggtc

SEQ ID NO: 67 - protein, 98 aa, Glycine max
  misc_feature: Ceres CLONE ID no. 651757
MVLDSILSSSPCLKSPSFSRQFARHELGSWSTLVKRHCFLLSALALLTVLCTIYLYFAVTFAANDSCSGLSGSLRDSCHMEHVMDSEAXSXLKGXRHL

SEQ ID NO: 68 - DNA, 527 bp, Arabidopsis thaliana
  misc_feature: Encodes the peptide sequence at SEQ ID NO. 69
aaatttaattctctctaatcaatttagttcctaaatatccggtttttgagaagaaacccgcgtttaccggcgaatcggctctttactctctagccggagacgagatctacaattttgacgactctgcctaagatatagcaattattgggagctgcacgaagaagattttatttggtcgacggagctggaatcagctattctttcttcttctccctaatcgtttcttttagcagctctttctctctcacccgaaggttggtgttaagctggaaaggttggtcttttagaagatatggttcttgatgggattgtatcatcaccattacggaggccacatgctcttaagaagcaatgggatgacttgggtagctgctctactgttgtccagaggcatcgtttccttttgactgctatgcttcttttggcattcctctgcaccatttatatctactttgccgtcacactaggcgctaggcacttgttgtgctcagggatgactggaaaagacaaggcaatgtgtcaaatgg

SEQ ID NO: 69 - protein, 78 aa, Arabidopsis thaliana
  misc_feature: Ceres CLONE ID no. 6932
MVLDGIVSSPLRRPHALKKQWDDLGSCSTVVQRHRFLLTAMLLLAFLCTIYIYFAVTLGARHLLCSGMTGKDKAMCQM